(19) United States
(12) Patent Application Publication
(10) Pub. No.: US 2006/0257395 A1
(43) Pub. Date: Nov. 16, 2006

(54) System and method for magnifying a humoral immune response

(75) Inventors: Muriel Y. Ishikawa, Livermore, CA (US); Edward K.Y. Jung, Bellevue, WA (US); Nathan P. Myhrvold, Medina, WA (US); Richa Wilson, San Francisco, CA (US); Lowell L. Wood JR., Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/131,155

(22) Filed: May 16, 2005

(51) Int. Cl. A61K 39/395; G06F 19/00; G06Q 50/00

(52) U.S. Cl. 424/133.1; 702/19; 705/3

(57) ABSTRACT

The present application relates, in general, to a system and/or method for detection and/or treatment.

### CROSS-REFERENCE TO RELATED APPLICATIONS

The present application is related to, claims the earliest available effective filing date(s) from (e.g., claims earliest available priority dates for other than provisional patent applications; claims benefits under 35 USC §119(e) for provisional patent applications), and incorporates by reference in its entirety all subject matter of the following listed application(s) (the “Related Applications”); the present application also claims the earliest available effective filing date(s) from, and also incorporates by reference in its entirety all subject matter of any and all parent, grandparent, great-grandparent, etc. applications of the Related Application(s) The United States Patent Office (USPTO) has published a notice to the effect that the USPTO's computer programs require that patent applicants reference both a serial number and indicate whether an application is a continuation or continuation in part. The present applicant entity has provided below a specific reference to the application(s)from which priority is being claimed as recited by statute. Applicant entity understands that the statute is unambiguous in its specific reference language and does not require either a serial number or any characterization such as “continuation” or “continuation-in-part.” Notwithstanding the foregoing, applicant entity understands that the USPTO's computer programs have certain data entry requirements, and hence applicant entity is designating the present application as a continuation in part of its parent applications, but expressly points out that such designations are not to be construed in any way as any type of commentary and/or admission as to whether or not the present application contains any new matter in addition to the matter of its parent application(s).

Related Applications:

1. For purposes of the USPTO extra-statutory requirements, the present application constitutes a continuation in part of currently co-pending United States patent application entitled A SYSTEM AND METHOD RELATED TO IMPROVING AN IMMUNE SYSTEM naming Muriel Y. Ishikawa, Edward K. Y. Jung, Nathan P. Myhrvold, Richa Wilson, and Lowell L. Wood, Jr. as inventors, filed 24 Aug. 2004 having U.S. application Ser. No. 10/925,904.
2. For purposes of the USPTO extra-statutory requirements, the present application constitutes a continuation in part of currently co-pending United States patent application entitled A SYSTEM AND METHOD FOR HEIGHTENING AN IMMUNE RESPONSE naming Muriel Y. Ishikawa, Edward K. Y. Jung, Nathan P. Myhrvold, Richa Wilson, and Lowell L. Wood, Jr. as inventors, filed 25 Aug., 2004 having U.S. application Ser. No. 10/926,753.
3. For purposes of the USPTO extra-statutory requirements, the present application constitutes a continuation in part of currently co-pending United States patent application entitled A SYSTEM AND METHOD RELATED TO AUGMENTING AN IMMUNE SYSTEM naming Muriel Y. Ishikawa, Edward K. Y. Jung, Nathan P. Myhrvold, Richa Wilson, and Lowell L. Wood, Jr. as inventors, filed 24 Aug., 2004 having U.S. application Ser. No. 10/925,905.
4. For purposes of the USPTO extra-statutory requirements, the present application constitutes a continuation in part of currently co-pending United States patent application entitled A SYSTEM AND METHOD RELATED TO ENHANCING AN IMMUNE SYSTEM naming Muriel Y. Ishikawa, Edward K. Y. Jung, Nathan P. Myhrvold, Richa Wilson, and Lowell L. Wood, Jr. as inventors, filed 24 Aug., 2004 having U.S. application Ser. No. 10/925,902.
5. For purposes of the USPTO extra-statutory requirements, the present application constitutes a continuation in part of currently co-pending United States patent application entitled A SYSTEM AND METHOD FOR MAGNIFYING AN IMMUNE RESPONSE naming Muriel Y. Ishikawa, Edward K. Y. Jung, Nathan P. Myhrvold, Richa Wilson, and Lowell L. Wood, Jr. as inventors, filed 25 Aug., 2004 having U.S. application Ser. No. 10/926,881.
6. For purposes of the USPTO extra-statutory requirements, the present application constitutes a continuation in part of currently co-pending United States patent application entitled A SYSTEM AND METHOD FOR MODULATING A HUMORAL IMMUNE RESPONSE naming Muriel Y. Ishikawa, Edward K. Y. Jung, Nathan P. Myhrvold, Richa Wilson, and Lowell L. Wood, Jr. as inventors, filed 1 Dec. 2004, having USAN [To Be Assigned by the PTO].
7. For purposes of the USPTO extra-statutory requirements, the present application constitutes a continuation in part of currently co-pending United States patent application entitled A SYSTEM AND METHOD FOR HEIGHTENING A HUMORAL IMMUNE RESPONSE naming Muriel Y. Ishikawa, Edward K. Y. Jung, Nathan P. Myhrvold, Richa Wilson, and Lowell L. Wood, Jr. as inventors, filed 3 Dec. 2004, having USAN [to be determined by USPTO]
8. For purposes of the USPTO extra-statutory requirements, the present application constitutes a continuation in part of currently co-pending United States patent application entitled A SYSTEM AND METHOD FOR AUGMENTING A HUMORAL IMMUNE RESPONSE naming Muriel Y. Ishikawa, Edward K. Y. Jung, Nathan P. Myhrvold, Richa Wilson, and Lowell L. Wood, Jr. as inventors, filed 3 Dec. 2004, having USAN [To be determined by the USPTO].
9. For purposes of the USPTO extra-statutory requirements, the present application constitutes a continuation in part of currently co-pending United States patent application entitled A SYSTEM AND METHOD FOR IMPROVING A HUMORAL IMMUNE RESPONSE naming Muriel Y. Ishikawa, Edward K.Y. Jung, Nathan P. Myhrvold, Richa Wilson, and Lowell L. Wood, Jr. as inventors, filed 26 Jan., 2005 having USAN [To be determined by the USPTO].

### TECHNICAL FIELD

The present application relates, in general, to detection and/or treatment.

### SUMMARY

In one aspect, a method includes but is not limited to: delivering a treatment to a host wherein the treatment is associated with modulating a predicted pattern of progression of one or more computable epitopes. In addition to the foregoing, other method aspects are described in the claims, drawings, and text forming a part of the present application.

In one aspect, a system includes but is not limited to: circuitry for suggesting delivery of a treatment to a host wherein the treatment is associated with modulating a predicted pattern of progression of one or more computable epitopes. In addition to the foregoing, other system aspects are described in the claims, drawings, and text forming a part of the present application.

In one aspect, a system includes but is not limited to: a computer readable medium including, but not limited to, a computer program for use with a computer system and wherein the computer program includes a plurality of instructions including one or more instructions for selecting one or more computable epitopes; one or more instructions for predicting at least one pattern change in the one or more computable epitopes; one or more instructions for associating the at least one pattern change in the one or more computable epitopes with at least one outcome; and one or more instructions for designating a course of action associated with the at least one pattern change in the one or more computable epitopes. In addition to the foregoing, other system aspects are described in the claims, drawings, and text forming a part of the present application.

In one aspect, a system includes but is not limited to: circuitry for selecting one or more computable epitopes; circuitry for predicting at least one pattern change in the one or more computable epitopes; circuitry for associating the at least one pattern change in the one or more computable epitopes with at least one outcome; and circuitry for designating a course of action associated with the at least one pattern change in the one or more computable epitopes. In addition to the foregoing, other system aspects are described in the claims, drawings, and text forming a part of the present application.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In addition to the foregoing, various other method and or system aspects are set forth and described in the text (e.g., claims and/or detailed description) and/or drawings of the present application.

The foregoing is a summary and thus contains, by necessity; simplifications, generalizations and omissions of detail; consequently, those skilled in the art will appreciate that the summary is illustrative only and is NOT intended to be in any way limiting. Other aspects, inventive features, and advantages of the devices and/or processes described herein, as defined solely by the claims, will become apparent in the non-limiting detailed description set forth herein.

### BRIEF DESCRIPTION OF THE FIGURES

FIG. 1 depicts a partial view of a system that may serve as an illustrative environment of and/or for subject matter technologies.

FIG. 2 depicts a partial view of a system that may serve as an illustrative environment of and/or for subject matter technologies.

FIG. 3 depicts a partial view of a system that may serve as an illustrative environment of and/or for subject matter technologies.

FIG. 4 depicts a diagrammatic view of one aspect of an exemplary interaction of an immune response component, for example, an antibody interacting with an epitope displayed by an agent.

FIG. 5 depicts a diagrammatic view of one aspect of a method of enhancing an immune response.

FIG. 6 depicts one aspect of an antigen-antibody interaction showing the occurrence of mutational changes in a selected epitope and corresponding changes in a complementary antibody.

FIG. 7 illustrates one aspect of mutational changes in an epitope displayed by an agent and the corresponding changes in an immune response component; for example, one or more new epitopes may appear on the surface of the agent.

FIG. 8 depicts a high-level logic flowchart of a process.

FIG. 9A depicts a high-level logic flowchart depicting alternate implementations of the high-level logic flowchart of FIG. 8.

FIG. 9B depicts a high-level logic flowchart depicting alternate implementations of the high-level logic flowchart of FIG. 8.

FIG. 9C depicts a high-level logic flowchart depicting alternate implementations of the high-level logic flowchart of FIG. 8.

FIG. 9D depicts a high-level logic flowchart depicting alternate implementations of the high-level logic flowchart of FIG. 8.

The use of the same symbols in different drawings typically indicates similar or identical items.

### DETAILED DESCRIPTION

The present application uses formal outline headings for clarity of presentation. However, it is to be understood that the outline headings are for presentation purposes, and that different types of subject matter may be discussed throughout the application (e.g., device(s)/structure(s) may be described under the process(es)/operations heading(s) and/or process(es)/operations may be discussed under structure(s)/process(es) headings). Hence, the use of the formal outline headings is not intended to be in any way limiting.

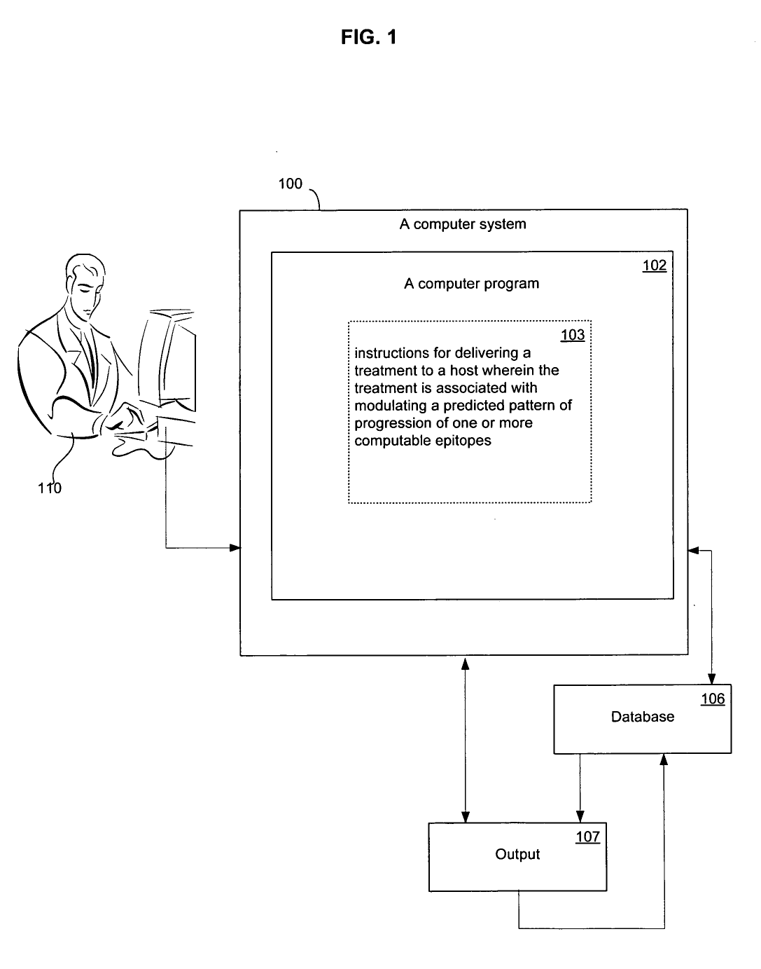

With reference now to FIG. 1, depicted is one aspect of a system that may serve as an illustrative environment of and/or for subject matter technologies, for example, a computer-based method for designating an immune response component for modulating an epitope and/or a computable epitope displayed by an agent. Accordingly, the present application first describes certain specific exemplary systems of FIG. 1; thereafter, the present application illustrates certain specific exemplary structures and processes. Those having skill in the art will appreciate that the specific structures and processes described herein are intended as merely illustrative of their more general counterparts. It will also be appreciated by those of skill in the art that an epitope-antibody, a computable epitope-antibody interaction, an immune cell receptor-epitope and/or immune-cell secretion product-epitope, and/or an antigen-antibody interaction is an exemplary interaction of an immune response component with an epitope, a computable epitope, and/or an antigen. Therefore, although, the exact nature of the interaction may vary, the overall picture as described herein and/or in other related applications relates to the interaction of an immune response component interacting with the epitope, computable epitope, and/or the antigen. As used herein, the term “epitope” 402 may, if appropriate to context, be used interchangeably with computable epitope, antigen, paratope binding site, antigenic determinant, and/or determinant.

A. Structure(s) and or System(s)

With reference to the figures, and with reference now to FIG. 1, depicted is a partial view of a system that may serve as an illustrative environment of and/or for subject matter technologies. One or more users 110 may use a computer system 100 including a computer program 102, for example, for providing and/or delivering a treatment associated with a disease, disorder, or condition. The computer program 102 may include one or more instructions, for example, instructions for delivering a treatment to a host wherein the treatment is associated with modulating a predicted pattern of progression of one or more computable epitopes 103. The instructions may be such that, when they are loaded to a general-purpose computer or microprocessor programmed to carry out the instructions, they create a new machine, because a general purpose computer in effect may become a special-purpose computer once it is programmed to perform particular functions pursuant to instructions from program software. That is, the instructions of the software program may electrically change the general-purpose computer by creating electrical paths within the device. These electrical paths, in some implementations, may create a special-purpose machine having circuitry for carrying out the particular program. The computer program 102 may include instructions that give rise to circuitry for delivering a treatment to a host wherein the treatment is associated with modulating a predicted pattern of progression of one or more computable epitopes 103. The treatment, may be provided based on, for example, including, but not limited to, information specific to the host and/or agent. In one aspect, the treatment, may include a plan and/or a protocol for treating a person. The treatment may include, and is not limited to, the treatment of a disease, disorder, condition, management of health in a healthy individual, and/or the management of health in an at-risk individual. Additionally, the protocol and/or plan of providing and/or delivering a treatment, may include, information relating to a route of deliverance, type of deliverable, agent to be eradicated, and/or program of treatment. The computer program 102 may accept input, for example, from medical personnel, a researcher, wet lab personnel, or equipment thereof. A user interface may be coupled to provide access to the computer program 102. In one implementation, the computer program 102 may access a database 106 storing information and transmit an output 107 to the computer system 100. In one exemplary implementation a feedback loop is set up between the computer program 102 and the database 106. The output 107 may be fed back into the computer program 102 and/or displayed on the computer system 100. The system may be used as a research tool, as a tool for furthering treatment or the like. This feedback scheme may be useful in an iterative process such as described herein and elsewhere.

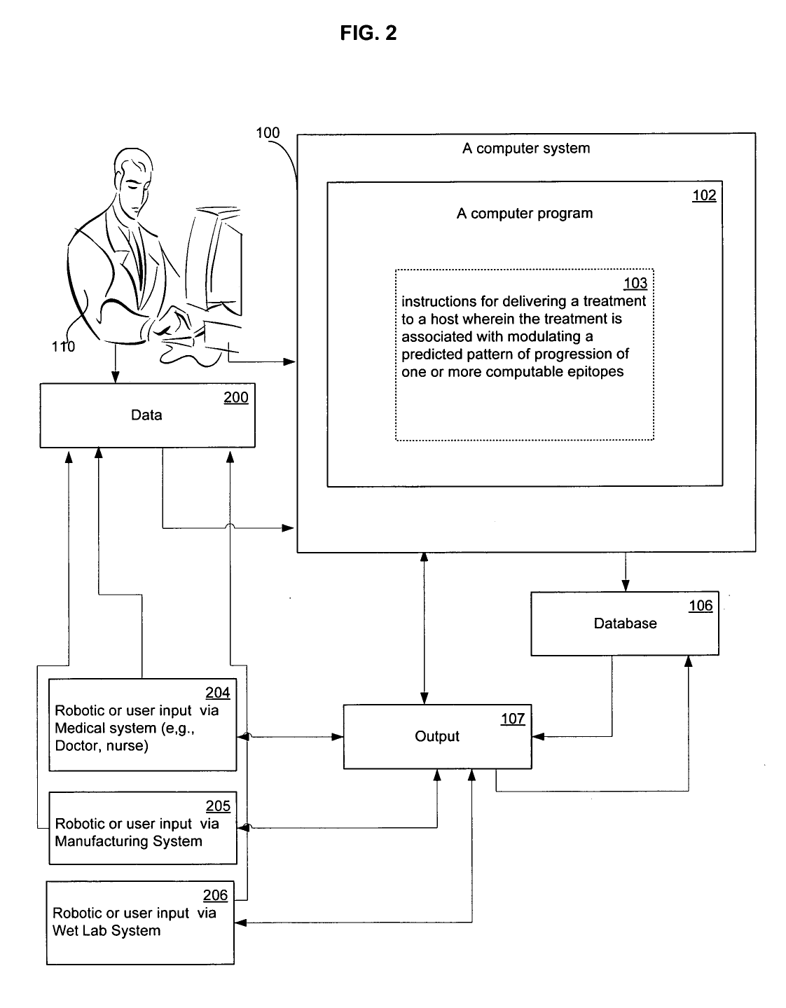

With reference to the figures, and with reference now to FIG. 2, depicted is a partial view of a system that may serve as an illustrative environment of and/or for subject matter technologies. The database 106, data 200, and/or the output 107 may be accessed by various input mechanisms, for example, mechanisms including but not limited to, robotic and/or user input via medical system 204, robotic and/or user input via manufacturing system 205, or robotic and/or user input via wet lab system 206. Access to the data 200 may be provided, for example, for further manipulation of the data.

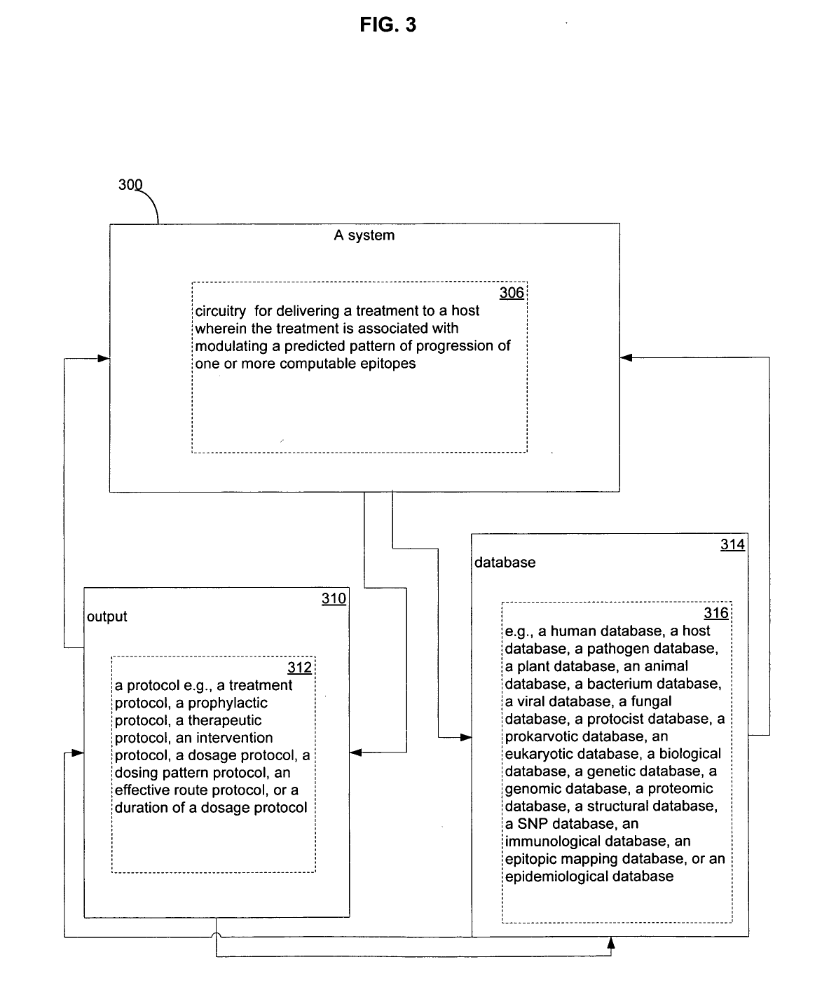

With reference to the figures, and with reference now to FIG. 3, depicted is a partial view of a system that may serve as an illustrative environment of and/or for subject matter technologies. In one aspect, a system 300 may include circuitry and/or components 304 for delivering a treatment to a host wherein the treatment is associated with modulating a predicted pattern of progression of one or more computable epitopes 306. Those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one of skill in the art in light of this disclosure.

Continuing to refer to FIG. 3, the system 300 may be coupled to a database 314 of an identifiable type 316, for example, including, but not limited to, a human database, a host database, a pathogen database, a plant database, an animal database, a bacterium database, a viral database, a fungal database, a protoctist database, a prokaryotic database, an eukaryotic database, a biological database, a genetic database, a genomic database, a proteomic database, a structural database, a SNP database, an immunological database, an epitopic mapping database, and/or an epidemiological database. An output 310 may be displayed, for example, in the form of a protocol 312, for example, including but not limited to a treatment protocol, a prophylactic protocol, a therapeutic protocol, an intervention protocol, a dosage protocol, a dosing pattern (in space, in time or in some combination thereof) protocol, an effective route protocol, and/or a duration of a dosage protocol. In one aspect, the type of output 310 may be selected by the user.

In various aspects, the computer system 100, the computer program 102 and/or the circuitry include predictive algorithms for determining the pattern changes in the computable epitope and the sequence of the computable epitope. In other various aspects, the computer system 100, the computer program 102 and/or the circuitry include predictive algorithms for determining the course of a disease influenced by the pattern changes in the computable epitope of the agent.

In various aspects, the computer system 100, the computer program 102 and/or the circuitry includes computer-based modeling software for designing and selecting the immune response component for reducing the ability of the agent to establish a significant presence in a host and/or to cause a disease, disorder and/or a condition that may require management.

In other various aspects, the computer system 100, the computer program 102 and/or the circuitry includes software for integrating with other computer-based systems and incorporating information relevant to selecting an immune response component for modulating the computable epitopes.

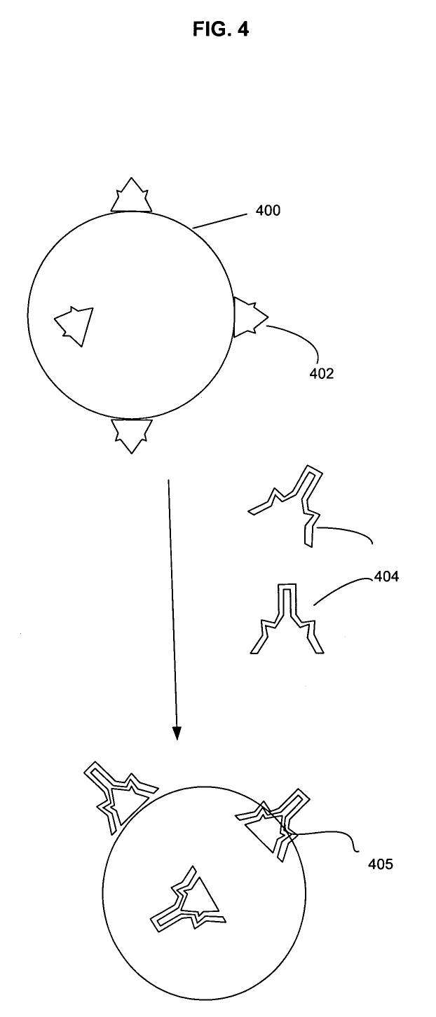

With reference to the figures, and with reference now to FIG. 4, depicted is a diagrammatic view of one aspect of an exemplary interaction of an immune response component, for example, an antibody 404 interacting with an epitope 402 displayed by an agent 400, for example, including but not limited to, in consequence of an interaction involving the agent 400.

The term “immune response component,” as used herein, may include, but is not limited to, at least a part of a macrophage, a neutrophil, a cytotoxic cell, a lymphocyte, a T-lymphocyte, a killer T-lymphocyte, an immune response modulator, a helper T-lymphocyte, an antigen receptor, an antigen-presenting cell, a dendritic cell, a cytotoxic T-lymphocyte, a T-8 lymphocyte, a cluster differentiation (CD) molecule, a CD3 molecule, a CD1 molecule, a B lymphocyte, an antibody, a recombinant antibody, a genetically engineered antibody, a chimeric antibody, a monospecific antibody, a bispecific antibody, a multispecific antibody, a diabody, a chimeric antibody, a humanized antibody, a human antibody, a heteroantibody, a monoclonal antibody, a polyclonal antibody, a camelized antibody, a deimmunized antibody, an anti-idiotypic antibody, an antibody fragment, a synthetic antibody, and/or any component of the immune system that may bind to an antigen and/or an epitope thereof in a specific and/or a useful manner. The term “agent,” as used herein, 400 may include, for example, but is not limited to, an organism, a virus, a dependent virus, an associated virus, a bacterium, a yeast, a mold, a fungus, a protoctist, an archaea, a mycoplasma, a phage, a mycobacterium, an ureaplasma, a chlamydia, a rickettsia, a nanobacterium, a prion, an agent responsible for a transmissible spongiform encephalopathy (TSE), a multicellular parasite, a protein, an infectious protein, a polypeptide, a polyribonucleotide, a polydeoxyribonucleotide, a polyglycopeptide, a nucleic acid, an infectious nucleic acid, a polymeric nucleic acid, a metabolic byproduct, a cellular byproduct, and/or a toxin. The term “virus” as used herein, may include, but is not limited to, a provirus, a retrovirus, a virus carrying oncogenes derived from normal cellular genes, and/or an oncovirus, unless context dictates otherwise. The term “agent” 400 may include, but is not limited to, a putative causative agent of a disease, a disorder, a cell, and/or component thereof that is deemed, for example, a target for therapy, a target for neutralization, and/or a cell whose removal, lysis or functional degradation may prove beneficial to the host. The term “agent” 400 may also include, but is not limited to, a byproduct or output of a cell that may be neutralized and/or whose removal or functional neutralization may prove beneficial to the host. Furthermore, the term “agent” 400 may include an agent belonging to the same family or group as the agent of primary interest, or an agent exhibiting a common and/or a biological function relative to the agent of primary interest.

The term “antibody” 404, as used herein, is used in the broadest possible sense and may include, but is not limited to, an antibody, a recombinant antibody, a genetically engineered antibody, a chimeric antibody, a monospecific antibody, a bispecific antibody, a multispecific antibody, a diabody, a chimeric antibody, a humanized antibody, a human antibody, a heteroantibody, a monoclonal antibody, a polyclonal antibody, a camelized antibody, a deimmunized antibody, an anti-idiotypic antibody, and/or an antibody fragment. The term “antibody” may also include, but is not limited to, types of antibodies such as IgA, IgD, IgE, IgG and/or IgM, and/or the subtypes IgG1, IgG2, IgG3, IgG4, IgA1 and/or IgA2. The term “antibody” may also include, but is not limited to, an antibody fragment such as at least a portion of an intact antibody 104, for instance, the antigen-binding variable region. Examples of antibody fragments may include Fv, Fab, Fab′, F(ab′), F(ab′).sub.2, Fv fragment, diabody, linear antibody, single-chain antibody molecule, multispecific antibody, and/or other antigen-binding sequences of an antibody. Additional information may be found in U.S. Pat. No. 5,641,870, U.S. Pat. No. 4,816,567, WO 93/11161, Holliger et al., Diabodies: small bivalent and bispecific antibody fragments, PNAS, 90: 6444-6448 (1993), Zapata et al., Engineering linear F(ab′)2 fragments for efficient production in Escherichia coli and enhanced antiproliferative activity, Protein Eng. 8(10): 1057-1062 (1995), which are incorporated herein by reference. Antibodies may be generated for therapeutic purposes by a variety of known techniques, such as, for example, phage display, and/or transgenic animals.

The term “antibody” 404, as used herein, may include anti-idiotypic antibodies. Anti-idiotypic antibodies may elicit a stronger immune response compared to the antigen and may be used for enhancing the immune response. Anti-idiotypic antibodies may be rapidly selected, for example, by phage display technology. Additional information may be found in U.S. Patent Application No. 20040143101, to Soltis, which is incorporated herein by reference.

The term “antibody” 404, as used herein, may include, but is not limited to, functional derivatives of a monoclonal antibody, which include antibody molecules or fragments thereof that have retained a dominant fraction of the antigenic specificity and/or the functional activity of the parent molecule.

The term “heteroantibody,” as used herein, may also include, but is not limited to, two or more antibodies, antibody fragments, antibody derivatives, and/or antibodies with at least one specificity that are linked together. Additional information may be found in U.S. Pat. No. 6,071,517, which is incorporated herein by reference.

The term “chimeric antibody,” as used herein, may include, but is not limited to antibodies having mouse-variable regions joined to human-constant regions. In one aspect, “chimeric antibody” includes antibodies with human framework regions combined with complementarity-determining regions (CDRs) obtained from a mouse and/or rat; those skilled in the art will appreciate that CDRs may be obtained from other sources. Additional information may be found in EPO Publication No 0239400, which is incorporated herein by reference.

The term “humanized antibody,” as used herein, may include, but is not limited to an antibody having one or more human-derived regions, and/or a chimeric antibody with one or more human-derived regions, also considered the recipient antibody, combined with CDRs from a donor mouse and/or rat immunoglobulin. In one aspect, a humanized antibody may include residues not found in either donor and/or recipient sequences. A humanized antibody may have single and/or multiple specificities. Additional information may be found in U.S. Pat. No. 5,530,101, and U.S. Pat. No. 4,816,567, which are incorporated herein by reference. Information may also be found in, Jones et al., Replacing the complementarity-determining regions in a human antibody with those from a mouse, Nature, 321:522-525(1986); Riechmann, et al., Reshaping human antibodies for therapy, Nature, 332:323-327 (1988); and Verhoeyen et al., Reshaping human antibodies: grafting an antilysozyme activity, Science, 239:1534 (1988), which are all incorporated herein by reference.

The term “human antibody,” as used herein may include, but is not limited to, an antibody with variable and constant regions derived from human germline immunoglobulin sequences. The term “human antibody” may include and is not limited to amino acid residues of non-human origin, encoded by non-human germline, such as, for example, residues introduced by site-directed mutations, random mutations, and/or insertions. Methods for producing human antibodies are known in the art and incorporated herein by reference. Additional information may be found in U.S. Pat. No. 4,634,666, which is incorporated herein by reference.

The term “recombinant antibody,” as used herein, may include antibodies formed and/or created by recombinant technology, including, but not limited to, chimeric, human, humanized, hetero antibodies and/or the like.

The term “epitope” 402, as used herein, may include, but is not limited to, a sequence of at least 3 amino acids, a sequence of at least nine nucleotides, an amino acid, a nucleotide, a carbohydrate, a protein, a lipid, a capsid protein, a polysaccharide, a sugar, a lipopolysaccharide, a glycolipid, a glycoprotein, and/or at least a part of a cell. As used herein, the term “epitope” 402 may, if appropriate to context, be used interchangeably with antigen, paratope binding site, antigenic determinant, and/or determinant. As used herein, the term “determinant” can include an influencing element, determining element, and/or factor, unless context indicates otherwise. In one aspect, the term “epitope” 402 includes, but is not limited to, a peptide-binding site. As used herein, the term “epitope” 402 may include structural and/or functionally similar sequences found in the agent 400. The term “epitope” 402 includes, but is not limited to, similar sequences observed in orthologs, paralogs, homologs, isofunctional homologs, heterofunctional homologs, heterospecific homologs, and/or pseudogenes of the agent 400. The epitope 402 may include any portion of the agent. In one aspect, the epitope 402 may include at least a portion of a gene or gene-expression product. In another aspect, the epitope may include at least a part of a non-coding region.

The term “computable epitope” as used herein, includes, but is not limited to, an epilope 402 whose likely future mutable forms may be predicted by using, for example, including but not limited to, practicable computer based predictive methodology and/or practicable evolutionary methods and/or practicable probabilistic evolutionary models and/or practicable probabilistic defect models and/or practicable probabilistic mutation models. For example, Smith et al. in their article “Mapping the Antigenic and Genetic Evolution of Influenza Virus” on the history of the antigenic evolution of the human influenza virus, Science 305, 371 (2004), which is incorporated herein by reference in its entirety, present in this paper's Table 1 and the supporting text thereof a set of patterns of viral coat-protein epitopic evolution which constitutes a basis for predicting one or more patterns of epitopic evolution in this particular agent, which is a well-established threat to human physiological well-being. In one aspect, the computable epitope may be suggested by, for example, including, but not limited to, predictive parallel extrapolations with similar structure, key residues, and/or the presence or absence of known domains. In another aspect, mathematics, statistical analysis and/or biological structural modeling tools may provide the relevant information for designating or identifying the computable epitope. One specific example of a computable epitope is a polypeptide associated with the HIV-1 virus, which may be, for example, seven to ten amino acids long. Knowing any starting state of such a polypeptide (e.g., how the various amino acids are sequenced/arranged), and using current computational techniques, it is practicable to calculate the likely future combinations of the seven to ten amino acids in the polypeptide so as to be able to predict how the epitope will likely appear as evolution/change occurs in the epitope, as biological processes take place. Indeed, many such evolutionary progressions in the protein sequences of the viral proteins (e.g., reverse transcriptase and protease) of the several major strains of HIV-1 virus have been reported in the literature, and are used for monitoring the clinical progression of disease in patients. Consequently, in some implementations, technologies described herein computationally predict how the epitope(s) will appear in future mutable forms. This predictive knowledge allows for the designation of at least one immune response component operable for modulating (e.g., reducing and/or eliminating) at least one “future version” of some posited presently existing epitope. As a specific example, one might predict the five or six mostly likely ways in which at least one epitope of a viral protein of a current strain of HIV-1 might appear a few months in the future, and then designate that a person's immune cells be exposed to the chemical structures of the epitopes of such an essential protein of such future HIV-1 strains to produce an immune response ready, waiting, and keyed to such future epitopic variants of the at least one HIV-1 strain. Once such antibodies or other immune response components have been produced, amplification or adjuvant techniques may be utilized to produce usefully-large quantities of such antibodies or other immune response components at a time earlier than the elapsing of the few months, and such antibodies administered to a host, or a vaccine eliciting such antibodies administered to a host, or cytotoxic responses prepared in the host, and/or a combination thereof. Then, if the HIV-1 virus does evolve or mutate in at least one of the five or six computationally-predicted ways, antibodies or other specific immune response components will be present and waiting to “lock onto” and negate the HIV-1 virus as it mutates along the predicted paths, thereby effectively precluding its ‘mutational escape’ from the initial therapy. Examples listed supra are merely illustrative of methodology that may be used for designating the computable epitope and are NOT intended to be in any way limiting.

Continuing to refer to FIG. 4, the epitope 402 or parts thereof may be displayed by the agent 400, may be displayed on the surface of the agent 400, extend from the surface of the agent 400, and/or may only be partially accessible by the immune response component. In one aspect, the epitope 402 may be a linear determinant. For example, the sequences may be adjacent to each other. In another aspect, the epitope 402 is a non-linear determinant, for example, including juxtaposed groups which are non-adjacent ab initio but become proximate to each other on folding or other assembly. Furthermore, the sequence of the non-linear determinant may be derived by proteasomal processing and/or other mechanisms (e.g., glycosolization, or the superficial ‘decoration’ of proteins with sugars) and the sequence synthetically prepared for presentation to the immune response component.

Continuing to refer to FIG. 4, in one aspect, the immune system launches a humoral response producing antibodies capable of recognizing and/or binding to the epitope 402 followed by the subsequent lysis of the agent 400. Mechanisms by which the antigen 402 elicits an immune response are known in the art and such mechanisms are incorporated herein by reference. In one aspect, the binding of the antibody 404 to the epitope 102 to form an antigen-antibody complex 405 is characterized as a lock-and-key fit. In another aspect, the binding affinity of the antibody (e.g., “best fit”) for the epitope may vary in time (e.g., in the course of ‘affinity maturation’ of the humoral immune response) or with physiological circumstances. In yet another aspect, the antigen-antibody complex may bind with varying degrees of reversibility. The binding or the detachment of the antigen-antibody complex may be manipulated, for example, by providing a small (e.g., relatively low molecular weight), (possibly solvated) atom, ion, molecule or compound that promotes the association or disassociation.

In one aspect, the epitope 402 is capable of evoking an immune response. The strength and/or type of the immune response may vary, for example, the epitope 402 may invoke a weak response and/or a medium response as measured by the strength of the immune response, e.g., as gauged by the peak antibody titer that results. It is contemplated that in one instance the epitope 402 selected for targeting may be one that invokes a weak response in the host; however, it may be selective to the agent 400. In another example, the epitope 402 selected may invoke a weak response in the host; however, it may be selected for targeting as it is common to a number of agents deemed to be targets. The herein-described implementations are merely exemplary and should be considered illustrative of like and/or more general implementations within the ambit of those having skill in the art in light of the teachings herein.

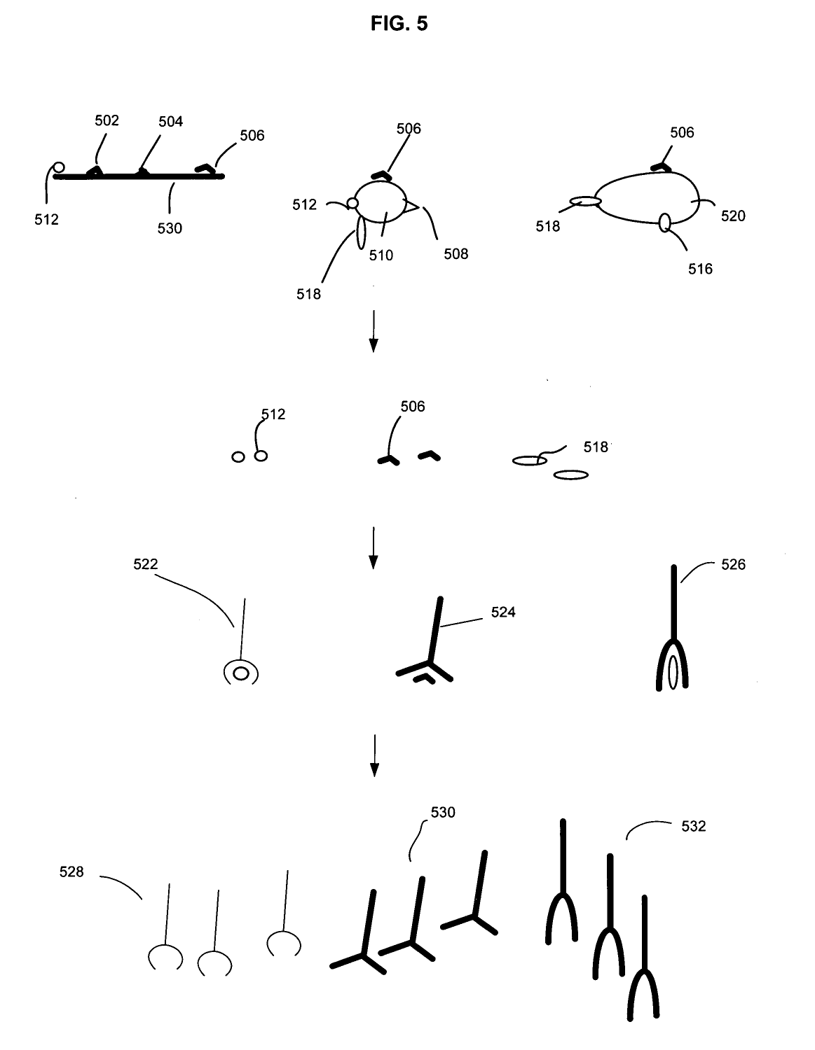

With reference to the figures, and with reference now to FIG. 5, depicted is a diagrammatic view of one aspect of a method of enhancing an immune response. In one aspect, an effective treatment therapy towards a disease and/or a disorder may utilize one or more immune response components designed to recognize one or more epitopes common to one or more agents. Such common or shared epitopes may represent an effective target group of epitopes. The immune response components designed to seek out and neutralize the common epitopes may be effective against one or more agents.

In one aspect, the one or more agents may be subtypes of the agent 400. In this aspect, a set of epitopes may be selected for targeting an agent. In another aspect, the one or more agents may be opportunistic agents capable of aiding or exaggerating an infection formed by the agent 400. In yet another aspect, the one or more agents may be agents known to establish a “beachhead” in the host organism prior to or subsequent to an infection or in response to the host's attenuated immune response.

With reference now to FIGS. 4 and 5, in one aspect, a shared epitope 506 is depicted as common to three agents 530, 510 and 520. In another aspect, a second shared epitope 512 is common to two agents 530 and 510. In yet another aspect, a third shared epitope 518 is common to two agents 510 and 520. Finding a subset of common epitopes shared amongst one or more agents may be done by statistical analysis, for example, by metaprofiling.

Continuing to refer to FIGS. 4 and 5, in one aspect, one or more agents 530, 510, and 520 depicted may share a subset of common epitopes. The selection of epitopes may depend on many different criteria. For example, the initial selection may be based on selection criteria including, but not limited to, the number of instances of presentation of the epitope 402 by one or more agents, the number of instances of presentation of the epitope 402 by the agent 400, the location of the epitope 402 in or on the agent, the size of the epitope 402, the nature of the epitope 402, the comparative sequence identity and/or homology of the epitope 402 with one or more host sequences, the composition of the epitope 402, and/or putative known or predicted changes in the epitope 402 sequence. The selection of epitopes may also depend on, for example, the type of immune response component desired for treating and/or managing the disease, disorder, and/or condition.

In one aspect, the epitope 402 selected has a probable sequence match with another agent of interest, for example, an opportunistic agent, or a subsequent prior, or concurrent infection caused by another agent. In another aspect, the epitope 402 selected has a low probable match with the host, for example, to decrease possible side effects due to the production of self- or auto-antibodies. The term “host,” as used herein, may include but is not limited to an individual, a person, a patient, and/or virtually any organism requiring management of a disease, disorder, and/or condition. For example, the epitope 402 selected may have a 0-70% sequence match at the amino acid level with the host or the agent 400, or a 0-100% sequence match with the agent. Those having skill in the art will recognize that part of that context in relation to the term “host” is that generally what is desired is a practicably close sequence match to the agent (e.g., HIV-1 or influenza-A virus), so that the one or more immune system components in use can attack it and a practicably distant sequence match to the host (e.g., a patient), in order to decrease or render less aggressive or less likely any attack on the host by the immune system components in use. However, it is also to be understood that, in some contexts, the agent will in fact constitute a part of the host (e.g., when the agent to be eradicated is actually a malfunctioning part of the host, such as in an auto-immune or neoplastic disease), in which case that part of the host to be eradicated will be treated as the “agent,” and that part of the host to be left relatively undisturbed will be treated regarded as the “host.” In another aspect, the epitope 402 selected has a sequence match with the agent, for example, a high degree of sequence match, or a relatively higher degree of sequence match with other agents compared to the host, or a 0-100% sequence match with the agent 400. The term “sequence match,” as used herein, includes sequence matching at the nucleic acid level, at the polysaccharide level, at the protein level, and/or at the polypeptide level. In an embodiment, the epitope 402 selected has a low percent sequence match with the host. In another embodiment, the epitope 402 selected has a high percent sequence match with other agents.

In molecular biology, the terms “percent sequence identity,” “percent sequence homology,” “percent sequence similarity,” or “percent sequence match” are sometimes used interchangeably. In this application, these terms are also often used interchangeably, with each other, and with “degree of sequence match,” unless context dictates otherwise.

In another aspect, the epitope 402 selected has a likely and/or a high percent sequence match with other epitopes, for example, including, but not limited to, the epitope 402 having a structural sequence match, a functional sequence match, a similar functional effect, a similar result in an assay and/or a combination. Structural comparison algorithms and/or 3-dimensional protein structure data may be used to determine whether two proteins or presented fragments thereof may have a usefully-high percent structural sequence match. In another example, the epitope 402 may have a functional match and/or share a similar functional effect with epitopes of interest. In this example, the epitope 402 may have a lower percent sequence match but may still exert the same functional effect. In another example, the epitope 402 and/or other epitopes of interest may have a lower percent sequence match but may share similar activities, for example, enzymatic activity and/or receptor binding activity, e.g., as determined by use of an assay.

In another aspect, the epitope 402 selected may be an immunological effective determinant, for example, the epitope 402 malt be weakly antigenic; however it may evoke an effective immune response deriving from, for example, the nature and/or the type of the immune response component that it induces. In another aspect, the epitope 402 may exert a similar effect on the immune response. For example, the epitope 402 selected may be part of the antigenic structure of an agent unrelated to the disease or disorder in question; however, it may exert a substantially similar effect on the immune system as assessed by, for example, the type, the nature, and/or the time-interval of the immune response induced thereby.

In one aspect, a sequence match with an entity may be determined by, for example, calculating the percent identity and/or percent similarity between epitopes and/or between the epitope 400 and the epitopic sequences of the host. In one aspect, the percent identity between two sequences may be calculated by determining a number of substantially similar positions obtained after aligning the sequences and introducing gaps. For example, in one implementation the percent identity between two sequences is treated as equal to (=) {a number of substantially similar positions÷total number of positions}×100. In this example, the number and length of gaps introduced to obtain optimal net alignment of the sequences is to be considered. In another aspect, the percent identity between two sequences at the nucleic acid level may be determined by using a publicly-available software tool such as BLAST, FASTA, BLAST-2, ALIGN and/or DNASTAR software. Similarly, the percent identity between two sequences at the amino acid level may be calculated by using publicly available software tools such as, for example, Peptidecutter, AACompSim, Find Mod, GlycoMod, InterProtScan, DALI and/or tools listed on the ExPasy Server (Expert Protein Analysis System) Proteomics Server at http://www.expasy.org/. In one embodiment, the percent identity at the nucleic acid level and/or at the amino acid level are determined.

In one aspect, string-matching algorithms may be used to identify homologous segments, for example, using FASTA and BLAST. In another aspect, sequence alignment based on fast Fourier transform (FFT) algorithms may be used to rapidly identify homologous segments. In yet another aspect, iterative searches may be used to identify and select homologous segment. Searches may be used not only to identify and select shared epitopes but also to identify epitopes that have the least homology with human sequences. Additional information may be found in Katoh, et al., MAFFT: a novel method for rapid multiple sequence alignment based on fast Fourier transform, Nucleic Acids Research, 30(14):3059-66 (2002) which is incorporated herein by reference.

A number of large-scale screening techniques may be used to identify and select the designed antibody, for example, the antibody designed may be selected by using optical fiber array devices capable of screening binding molecules. Additional information may be found in U.S. Patent Application No. 20040132112 to Kimon et al., which is hereby incorporated by reference.

It will be appreciated by those skilled in the art that the epitope 402 selected need not be limited to a matching sequence displayed by the agent 400. In one aspect, a meta-signature and/or a consensus sequence may be derived based on any number of criteria. In one aspect, the meta-signature may be derived by analysis of data from sources such as, for example, antigenic evolution, genetic evolution, antigenic shift, antigenic drift, data from crystal structure, probable match with a host, probable match with other agent strains, and/or strength of the immunogenic response desired. The meta-signature may include new sequences and/or may exclude some sequences. For example, it may include silent mutations, mismatches, a spacer to bypass a hotspot or a highly mutable site, predicted changes in the sequence, and/or may include epitopes from multiple agents, thus providing immune-based protection from multiple agents. As another example, the meta-signature may exclude sequences, such as, for example, including, but not limited to, mutable sequences and/or sequences with a high percent sequence match to the host's epitopes.

In one aspect, the predicted changes in the epitope 402 may be determined by analysis of past variations observed and/or predicted in the agent 400 (e.g., FIG. 5). Computational analysis can be used to determine regions showing sequence variations and/or hot spots. In one aspect, high speed serial passaging may be performed in silico, computationally mimicking the serial passaging that occurs naturally with a production of a new strain of the agent 400. It will be appreciated by those of skill in the art that the hot spots need not be identified by examining the epitope 402, and/or by examining the epitope 402 in context with the agent 400. Information pertaining to hot spots can also be extrapolated by performing sequence analysis of other agents and/or domain analysis of such other agents. For example, in one implementation, the epitope 402 may be part of a domain shared between multiple agents, some of which may lack the epitope 402 of interest. Information pertaining to hot spots identified in the domain of the other agents may be of practical use in determining the meta-signature.

In one aspect, one or more sets and/or subsets of epitopes may be formed. The nature and type of criteria used to form the sets and/or subsets will depend, for example, on the nature and type of the agent 400, the duration of the immune response desired (e.g., short-term immunity, or long-term immunity), the nature of the immune response desired (e.g., weak, moderate, or strong), the population to be protected (e.g., presence and/or currency of varying degrees of prior exposure) and the like. The sets and/or subsets so formed may accept input either robotically or from a user (e.g., from a manufacturer of immune response components, from a researcher or from a wet lab, and/or from medical personnel).

The pattern changes predicted in the epitope 402 may be supplemented, for example, by other methodology, statistical analysis, historical data, and/or other extrapolations of the type utilized by those having skill in the art. The knowledge of these predicted pattern changes represents an asset in the design and/or selection of the immune response components. The predicted pattern changes may be associated with an outcome (e.g., used to determine the progression of the changes in the immune response component required to manage such changes). In some implementations an outcome may be an estimated and/or a heuristic outcome. Inferring the pattern changes in the epitope 402 and using the information to modulate the progressing response may help manage the response more effectively. For example, the pattern changes may be used to provide a timeline of when the therapy might aptly be changed, what therapy should constitute the change, or the duration of the change. As a more specific example, one reason why HIV-1 virus is able to eventually kill its host is that the virus mutates its antigenic signature-profile significantly faster than the human immune system can effectively track and respond to these mutations. In a specific implementation of the subject matter described herein, a sample of a HIV-1 viral quasispecies is taken from a patient at a point in time and computational biological techniques are used to infer likely mutations of the antigenic signature-profile of the virus (e.g., viral quasispecies) at future times. Techniques such as cloning are then utilized to synthesize immune system-activating aspects of the anticipated future sub-strains of the virus (e.g., viral quasispecies), and thereafter replicative techniques are utilized to rapidly generate copious amounts of one or more immune system components (e.g., antibodies) that are keyed to the likely future generations (e.g., mutational variants) of the patient's particular strain and sub-strain(s) of HIV-1. Once prepared, the immune system components are then administered to the patient and thus are “present and waiting” for the HIV-1 viral quasispecies components when it mutates into the anticipated new forms and/or attempts to proliferate these forms. If the HIV-1 quasi-species mutates as anticipated, the preloaded immune response components successfully negate the mutated quasi-species components, thereby likely greatly reducing the patient's viral load—and crucially suppressing the likelihood of further mutation, since the virion population of mutated forms never becomes substantial. In another implementation, the mutational history of the HIV-1 quasispecies is closely tracked, and once the actual mutational direction has been determined, high-speed (likely, ex vivo) techniques are utilized to generate immune system components capable of effective suppression of the mutated viral quasispecies, significantly more rapidly than the virus is able to effectively mutate and thus ‘escape’ from the suppressive therapy.

In one aspect, the epitope 402 selected for designating the immune response component may be synthetically made and/or derived from the agent 400. In one embodiment, the epitope 402 selected is derived from an agent 400 extracted from an individual desiring treatment and/or an individual found to be resistant to that agent. In one aspect, the epitope 402 selected for designating the immune response component may include multiple copies of the exact same epitope and/or multiple copies of different epitopes.

In one aspect, the meta-signature includes sequences matching adjacent and/or contiguous sequences. In another aspect, the meta-signature includes non-adjacent sequences. For example, it will be appreciated by those of skill in the art that peptide splicing and/or proteosomal processing of the epitope 402 that occurs naturally may result in the formation of a new epitope, for example, a non-linear epitope. In this example, proteosomal processing may result in the excision of sequences and the transposing and/or juxtaposing of non-contiguous sequences to form the non-linear epitope. Additional information may be found in Hanada et al., Immune recognition of a human renal cancer antigen through post-translational protein splicing, Nature 427:252 (2004), and Vigneron et al., An antigenic peptide produced by peptide splicing in the Proteosome, Science 304:587 (2004), hereby incorporated by reference herein in their entireties.

Additionally, it will also be appreciated by those of skill in the art that the meta-signature may include sequences displayed on two different parts of the agent 400. For example, non-adjacent sequences may appear adjacent to each other when the protein is folded. In this aspect, the meta-signature may include the non-adjacent sequences for identifying the meta-signature. Furthermore, the meta-signature may include non-adjacent sequences corresponding to a specific conformational state of a protein. Immune response components designed to bind such sequences may be specific to the conformational state of the protein. 3-D and/or crystal structure information may also be used to designate the meta-signature.

In one aspect, the meta-signature may include multiple sets of epitopes targeting a predicted pattern change and/or an observed pattern change. For example, multiple sets of epitopes may be designed for vaccination and/or for production of immune response components.

Techniques for epitope mapping are known in the art and herein incorporated by reference. For example, FACS analysis and ELISA may be used to investigate the binding of antibodies to synthetic peptides including at least a portion of the epitope. Epitope-mapping analysis techniques, Scatchard analysis and the like may be used to predict the ability of the antibody 404 to bind to the epitope 402 presented on the agent 100, to determine the binding affinity of the antibody 404 or other immune response component to the epitope 402, and/or to discern a desirable configuration for the antibody 404 or other immune response component.

Continuing to refer to FIG. 5, in one aspect, for example, the sequences of selected epitopes 506, 512, and/or 518 may be used to design one or more complementary antibodies 524, 522, and/or 526, or other immune response components, respectively. The sequences of selected epitopes 506, 512, and 518 may be used to form monoclonal antibodies, for example, by cloning or by using human-mouse systems.

The sequences of selected epitopes 506, 512, and/or 518 may be amplified using the polymerase chain reaction (PCR) as described in U.S. Pat. Nos. 4,683,195, 4,683,202, and 4,800,159 to Mullis et al. which are incorporated herein in their entirety. In another aspect, a consensus sequence and/or a meta-signature may be designed and amplified. The relevant sequence(s) may be inserted in an expression vector for producing proteins and the expressed protein(s) subsequently used to produce antibodies specific to the selected epitopes. In one aspect, the selected epitopes may be antigenic but may not be directly immunogenic.

Human antibodies may be made, for example, by using a human-mouse system such as, for example, the Xenomouse technology of Abgenix, Inc., (available from Abgenix, Inc. currently having corporate headquarters in Fremont, Calif. 94555) and/or the HuMAb Mouse technology of Medarex, Inc., (available from Medarex Inc. currently having corporate headquarters in Annadale, N.J.). In these systems, the host mouse immunoglobulins genes are inactivated and human immunoglobulin genes are inserted in the host. On stimulation with an antigen, such transgenic mice produce fully human antibodies. Subsequently, human monoclonal antibodies can be isolated according to standard hybridoma technology.

Selection of humanized antibodies with higher binding affinities from promising murine antibodies may be performed by using computer modeling software developed by Queen et al. The antibodies produced by this method include approximately 90% of the pertinent human sequences. The structure of the specific antibody is predicted based on computer modeling and the retaining of key amino acids predicted to be necessary to retain the shape and, therefore, the binding specificity of the complementarity determining regions (CDRs). Thus, key murine amino acids are substituted into the human antibody framework along with murine CDRs. The software may then be used to test the binding affinity of the redesigned antibody with the antigen. Additional information can be found in U.S. Pat. No. 5,693,762 to Queen et al., which is incorporated herein by reference.

The formation of other antibody fragments, such as, for example, Fv, Fab, F(ab′).sub.2 or Fc may be carried out by, for example, phage antibody generated using the techniques as described in McCafferty et al., Phage antibodies: filamentous phage displaying antibody variable domains, Nature 348:552-554 (1990), and Clackson et al., Making Antibody Fragments Using Phage Display Libraries, Nature 352:624-628 (1991) and U.S. Pat. No. 5,565,332 to Hoogenboom et al., which are incorporated herein by reference. Surface plasmon resonance techniques, for instance, may be used to analyze real-time biospecific interactions. Camelized antibodies, deimmunized antibodies and anti-idiotypic antibodies may be selected by techniques known in the art, which are herein incorporated by reference.

In one aspect, the selection of antibodies for modulating the immune response may be based on their function. For example, activating antibodies, blocking antibodies, neutralizing antibodies, and/or inhibitory antibodies may be used to modulate the immune response. Such antibodies may perform one or more functions under the appropriate conditions. In a more specific example, the antibody 404 may be triggered to undergo a conformational change by providing a cofactor and/or by changing the ambient temperature or other ambient conditions, such as overall osmolality or pH or concentration of a particular compound, atom or ion. The conformation change may result in a new function being performed by the antibody 404.

Techniques for purifying antibodies are known in the art and are incorporated herein by reference. The purified complementary antibodies 530, 528, or 532 may then be made available for therapeutic and/or prophylactic treatment.

The term “an effective treatment therapy,” as used herein, includes, but is not limited to, the use of immune response components in combination with other antibodies, antibody fragments, and/or in combination with other treatments, including, but not limited to, drugs, vitamins, hormones, medicinal agents, pharmaceutical compositions and/or other therapeutic and/or prophylactic combinations. In another aspect, the immune response component may be used in combination, for example, with a modulator of an immune response and/or a modulator of an antibody. In one aspect, cocktails of immune response components may be administered, for example, by injecting or otherwise effectively inserting by a subcutaneous, nasal, intranasal, intramuscular, intravenous, intraarterial, intrathecal, intracapsular, intraorbital, intracardiac, transdermal, subdermal, intradermal, intraperitoneal, transtracheal, subcuticular, intraarticular, subcapsular, subarachnoidal, intraspinal, epidural, intrasternal, infusion, topical, sublingual, and/or enteric route.

The therapeutic effect of the immune response component may be produced by one or more modes of action. For example, in one aspect, the immune response component may produce a therapeutic effect and/or alleviate the symptoms by targeting specific cells and neutralizing them. In another aspect, the immune response component may bind to and/or block receptors present on the agent 400 and/or may directly and/or indirectly block the binding of molecules, such as, for example, cytokines, and/or growth factors, or modulators or pro- or anti-apoptotic signaling materials to the agent 400. In another aspect, the therapeutic effect of the immune response component is produced by functioning as signaling molecule(s). In this example, the immune response component(s) may induce cross-linking of receptors with subsequent induction of programmed cell death (e.g., apoptosis).

The immune response component may be engineered to include, for example, one or more effector molecules, such as, for example, drugs, small molecules, enzymes, toxins, radionuclides, cytokines, and/or DNA molecules. In this example, the immune response component may serve as a vehicle for targeting and binding the agent 400 and/or delivering the one or more effector molecules. In one aspect, the immune response component may be engineered to include the one or more effector molecules without the natural effector functions of the immune response component.

In another aspect, one or more immune response components may be coupled to molecules for promoting immune system components to eliminate unwanted cells or other biological entities, such as virus particles. This technique has been described for the treatment of tumors, viral-infected cells, fungi, and bacteria using antibodies. Additional information may be found in U.S. Pat. No. 4,676,980 to Segal, which is incorporated herein by reference.

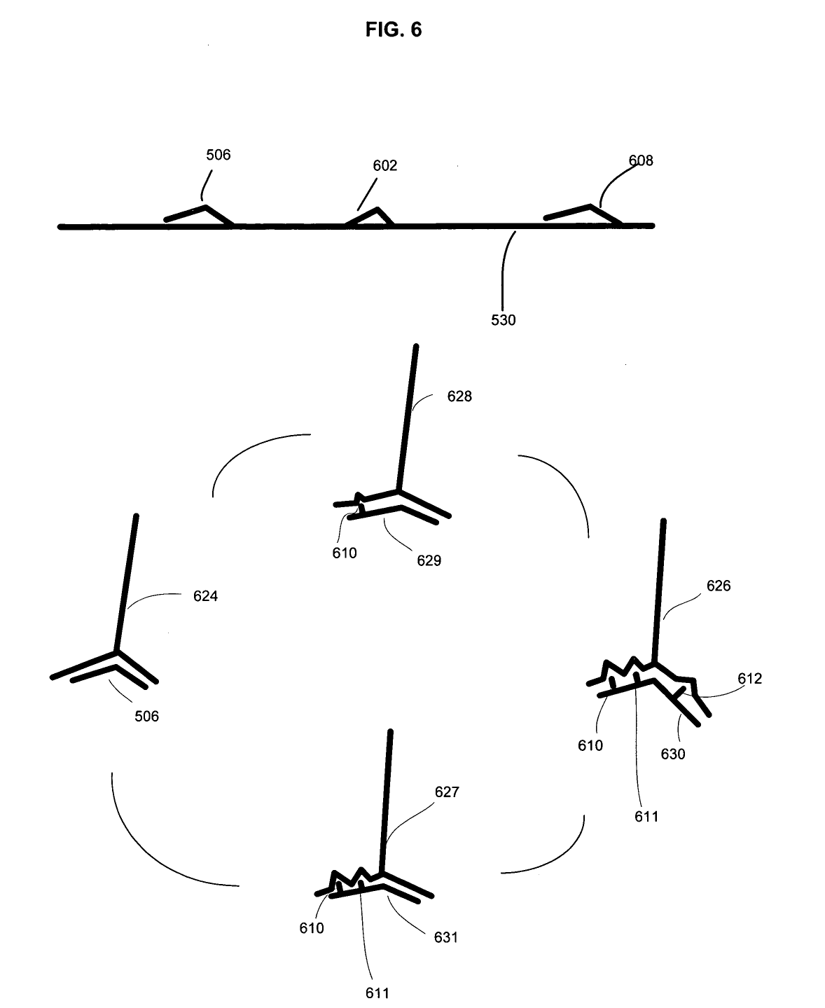

With reference to the figures, and with reference now to FIG. 6, depicted is one aspect of an antigen-antibody interaction showing the occurrence of mutational changes in a selected epitope and corresponding changes in a complementary antibody. The selected epitope 506 may undergo mutational changes. Other epitopes 602 and/or 608 may not be selected, for example, as the mutation rate for these epitopes may be substantially higher. These mutations may be random and, therefore, non-predictable, or they may be predictable. For example, a mutation may be substantially more predictable based on the occurrence of hot spots or known mutational history. The complementary antibody 624 or other immune response component may bind the selected epitope 506, for example, with a usefully-high affinity. However, a sequence change 610 depicted in a mutated selected epitope 629 may reduce the binding affinity of the complementary antibody or other immune response component 624. A complementary antibody or other immune response component incorporating the mutation 628 may restore the binding affinity, for example, to a usefully-high binding affinity. Similarly, appearance of mutations 610, 611 and 612 may require a new complementary antibody 626 or other immune response component in order to attain a usefully-high binding affinity. Additionally, the appearance of mutations 610 and 611 may require a new complementary antibody 627 or other immune response component. The predictive aspect of the computer system, software and/or circuitry may be used to make mathematically predictable hypotheses regarding the variations and the treatment components required. In one aspect, the complementary antibody or other immune response component need not have a high binding affinity. For example, the new antibody 626 or other immune response component may be used to bind and modulate the agents with mutations 610, 611 and/or 612.

In another aspect, the antibodies or other immune response components with higher binding affinities may be selected. Numerous techniques exist for enhancing the binding affinity of the antibody or other immune component for the epitope 402. In one aspect, the binding affinity of the antibody or other immune response component for the epitope 402 may be enhanced by constructing phage display libraries from an individual who has been immunized with the epitope 402 either by happenstance or by immunization. The generation and selection of higher affinity antibodies or other immune response components may also be improved, for example, by mimicking somatic hypermutagenesis, complementarity-determining region (CDR) walking mutagenesis, antibody chain shuffling, and/or technologies such as Xenomax technology (available from Abgenix, Inc. currently having corporate headquarters in Fremont, Calif. 94555). In one example, antibodies including introduced mutations may be displayed on the surface of filamentous bacteriophage. Processes mimicking the primary and/or secondary immune response may then be used to select the desired antibodies, for example, antibodies displaying a higher binding affinity for the antigen, and/or by evaluating the kinetics of dissociation. For additional information see, Low et al., Mimicking Somatic Hypermutation: Affinity Maturation Of Antibodies Displayed On Bacteriophage Using A Bacterial Mutator Strain, J. Mol. Biol. 260:359-368 (1996); Hawkins et al. Selection Of Phage Antibodies By Binding Affinity. Mimicking Affinity Maturation, J. Mol. Biol. 226:889-896 (1992), which are incorporated herein by reference.

In another example, the generation and/or selection of higher affinity antibodies may be carried out by CDR walking mutagenesis, which mimics the tertiary immune selection process. For example, saturation mutagenesis of the CDRs of the antibody 404 may be used to generate one or more libraries of antibody fragments which are displayed on the surface of filamentous bacteriophage followed by the subsequent selection of the relevant antibody using immobilized antigen. Sequential and parallel optimization strategies may be used to then select the higher affinity antibody. For additional information see Yang et al., CDR Walking Mutagenesis For The Affinity Maturation Of A Potent Human Anti-HIV-1 Antibody Into The Picomolar Range, J. Mol. Biol 254(3):392-403 (1995), which is incorporated herein by reference in its entirety.

In yet another example, site-directed mutagenesis may be used to generate and select higher affinity antibodies, for example, by parsimonious mutagenesis. In this example, a computer-based method is used to identify and screen amino acid residues included in the one or more CDRs of a variable region of an antibody 104 involved in an antigen-antibody binding. Additionally, in some implementations, the number of codons introduced is such that about 50% of the codons in the degenerate position are wild-type. In another example, antibody chain-shuffling may be used to generate and select higher affinity antibodies. These techniques are known in the art and are herein incorporated by reference.

The dosage of the immune response component may vary and in one aspect may depend, for example, on the duration of the treatment, body mass, history and/or severity of the disease, health-history, genotype, sex and/or age. Compositions including immune response components may be delivered to an individual for prophylactic and/or therapeutic treatments. In one aspect, an individual having a disease and/or condition is administered a treatment dose to alleviate and/or at least partially cure the condition manifested by the symptoms. In this example, a therapeutically-effective dose is administered to the patient.

In another aspect, a person's resistance to disease conditions may be enhanced by providing a prophylactically calibrated dose of the antibody 404. A prophylactic dose may be provided to, for example, including, but not limited to, a person genetically predisposed to a disease and/or condition, a person (about to be) present in a region where a disease is prevalent, and/or a person wishing to enhance that person's immune response.

Optimization of the physico-chemical properties of the immune response component may be improved, for example, by computer-based screening methods. Properties affecting antibody therapeutics may also be improved, such as, for example, stability, antigen binding affinity, and/or solubility. Additional information may be found in U.S. Patent Application No. 20040110226 to Lazar, which is incorporated herein by reference.

With reference to the figures, and with reference now to FIGS. 4, 5, and 6, depicted is one aspect of the antigen-antibody interaction showing the occurrence of mutational changes in the selected epitope 506 and corresponding changes in the complementary antibody or other immune response component 524. Such mutational changes in the selected epitope 506, for example, may be minor or major in nature. These minor and/or major antigenic variations may render an existing treatment less effective. Thus an effective treatment therapy towards a disease or disorder may include treating the disease or disorder with one or more antibodies designed to anticipate one or more predictable antigenic variations, for example, including, but not limited to, of one or more agents or one or more related agents, and/or shared with at least two agents. Furthermore, predicting the course of the minor and/or major antigenic variations of the agent 400 and/or the related agents would also be beneficial in designing or selecting these anticipatory antibodies. Additionally, in some implementations, the inclusion of information from SNP databases may be useful in helpful in designing antibodies for binding the selected epitope 506.

Minor changes in the epitope 402 which do not always lead to the formation of a new subtype may be caused, for example, by point mutations in the selected epitope 506. In one aspect, the occurrence of point mutations may be localized, for example, to hot spots of the selected epitope 506. The frequency and/or occurrence of such hot spots may be predicted by the computer-based method. Additionally, the method provides for access to databases including, for example, historical compilations of the antigenic variations of the agent 400 and/or of the selected epitope 506, for example, from previous endemics and/or pandemics or the natural evolutionary history of the disease. Such information may be part of an epitope profile for charting the progression of the immune response. A non-exclusive example is provided by a point mutation relative to the glutamic acid at position 92 of the NS1 protein of the influenza-A virus that has been shown to dramatically down-regulate activation of human cytokines. Such information may be useful in designating the meta-signature.

Continuing to refer to FIGS. 4, 5, and 6, depicted is that a mutation 610 in the selected epitope 506 results in a mutated epitope 629. The term “the selected epitope 506” as typically used herein, often constitutes a type of the more general term of presented epitope, unless context indicates otherwise. The generation of the mutated epitope 629 may reduce the binding of the immune response component, for example, the antibody 624. In one aspect, binding could be enhanced by generating a new antibody 628 corresponding to the mutated epitope 610. The frequency of minor antigenic variations may be predicted by examining known and/or predicted mutational hot spots. For example, additional mutations 611 and/or 612 may be predicted by the computer-based method and corresponding antibodies 626 and/or 627, respectively, may be designed to compensate for such antigenic variations in the mutated epitopes 630 and/or 631, respectively. In one aspect, an effective treatment therapy may incorporate this knowledge in providing an effective humoral immune response towards the agent 400. For example, a cocktail of immune response components may include the antibodies 624, 628, 626, and/or 627 for binding to the selected epitope 506 and/or its predicted mutated versions. In one aspect, the cocktail of one or more antibodies or other immune response components may be supplemented by additional chemicals, drugs, and/or growth- or replication- or survival-modulating factors. In another aspect, the effective treatment therapy may include varying doses of immune response components, for example, a substantially larger or more prolonged or earlier- or later-administered dosage of 626 relative to 624, 628, and/or 627.

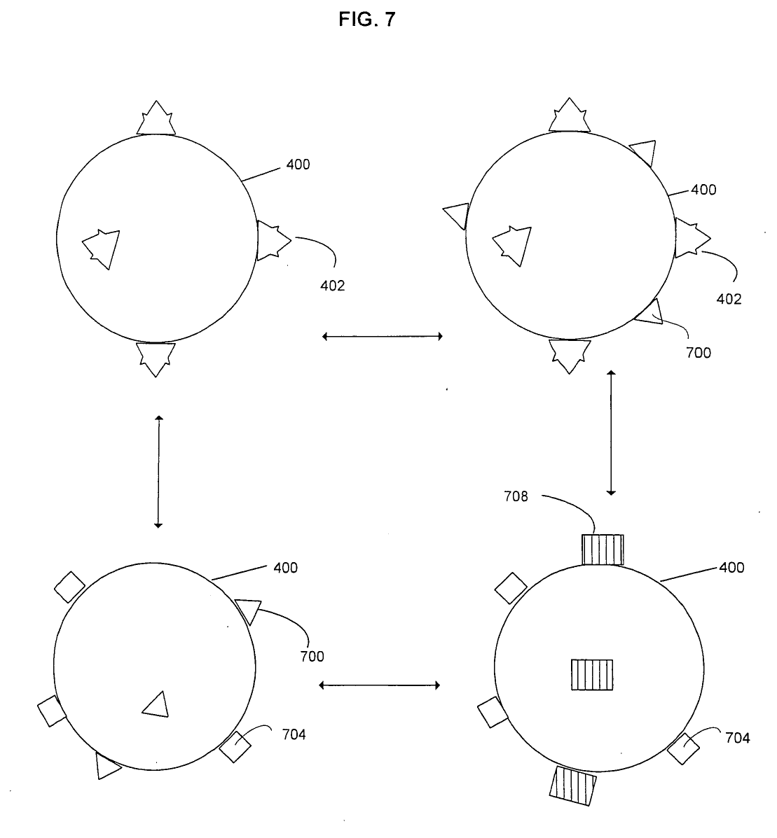

Referring now to FIG. 7, illustrated is one aspect of mutational changes in an epitope displayed by an agent and the corresponding changes in an immune response component, for example, one or more new epitopes 700 and/or 704 may appear on the surface of the agent 400. In one aspect, major changes may occur in the antigenic variants present on the surface of the agent 400 resulting in the formation of a new subtype or sub-strain. The appearance of new epitopes observed, for example, may occur as a result of antigenic shifts, reassortment, reshuffling, rearrangement of segments, and/or swapping of segments and sometimes marks the appearance of a new virulent and/or pathogenic (sub-)strain of the agent 400. In one instance, the prediction of the new epitopes may mark the emergence of a new (sub-)strain, a new subtype, and/or the reemergence of an older (sub-)strain. In this instance, natural and/or artificial immune protection in an individual alone may not provide adequate protection against initial infection or infection-progression. Immune protection and/or humoral protection may be supplemented with, for example, drugs, chemicals or small molecules capable of enhancing, supplanting or favorably interacting with the effects of the pertinent immune response components.

Generally, when major epitopic changes do occur, a larger fraction of the exposed host population becomes susceptible to infection by the agent, sometimes leading to an epidemic or a pandemic. This problem may be alleviated in part, for example, by predicting the appearance of new (sub-)strains and/or subtypes as a result of the appearance of new epitopes and/or the disappearance of existing epitopes. In one aspect, for example, including, but not limited to, the prediction of the new epitopes, attention may be directed towards a subset of genes, for example, important for the overall Darwinian fitness and/or replication and/or infectivity of the agent 400. For example, examining the appearance of new subtypes of influenza virus type A shows that the antigenic variations occur for the most part as a result of mutations in this virus's neuraminidase and/or hemagglutinin genes.

In another aspect, the selected epitope 506 may not involve highly variable regions and may focus instead on areas having lower probability of mutations. Thus epitopes selected may circumvent hot spots of antigenic variations and instead target other specific regions of the agent 400, such as, for example, the receptor-binding site on the surface of the agent 400. In another example, the selected epitope 506 may not be readily accessible to the immune response component, for example, the receptor-binding site may be buried deep in a ‘pocket’ of a large protein and may be surrounded by readily accessible sequences exhibiting higher level(s) of antigenic variations. In this example, one possibility may include providing small antibody fragments that penetrate the receptor-binding site preventing the agent 400 from binding its target. In another example, a drug and/or chemical may be used to modify and/or enhance the accessibility of the receptor-binding site. In yet another example, a chemical with a tag may be used to bind to the receptor and the tag then used for binding the immune response component.

In another aspect, the immune response component may be designed so as to circumvent the shape changes in the epitope 402 and provide sufficiently effective binding to the epitope 402 even following mutational change therein. In this example, the antibody or other immune response component designed may include accommodations in its design deriving from the prediction of hot spots and/or the predicted mutational changes in the epitope 402.

In one aspect, the size of the immune response component may be manipulated. An immune response component, for example the antibody 404, may be designed to include the practicably minimal binding site required to bind the epitope 402. In another example, the immune response component may be designed for binding to the practicably smallest effective determinant (e.g., of an epitope).

In one aspect, an effective treatment therapy for a disease and/or disorder may include one or more immune response components designed to anticipate and/or treat an antigenic drift and/or an antigenic shift predicted for multiple agents. The agents need not be related to each other; for example, the therapy might be designed for an individual suffering from multiple diseases (e.g., distinct infection-mediated diseases).

B. Operation(s) and/or Process(es)

Following are a series of flowcharts depicting implementations of processes. For ease of understanding, the flowcharts are organized such that the initial flowcharts present implementations via an overall “big picture” or “top-level” viewpoint and thereafter the following flowcharts present alternate implementations and/or expansions of the “big picture” flowcharts as either sub-steps or additional steps building on one or more earlier-presented flowcharts. Those having skill in the art will appreciate that the style of presentation utilized herein (e.g., beginning with a presentation of a flowchart(s) presenting an overall view and thereafter providing additions to and/or further details in subsequent flowcharts) generally allows for a more rapid and reliable understanding of the various process implementations.

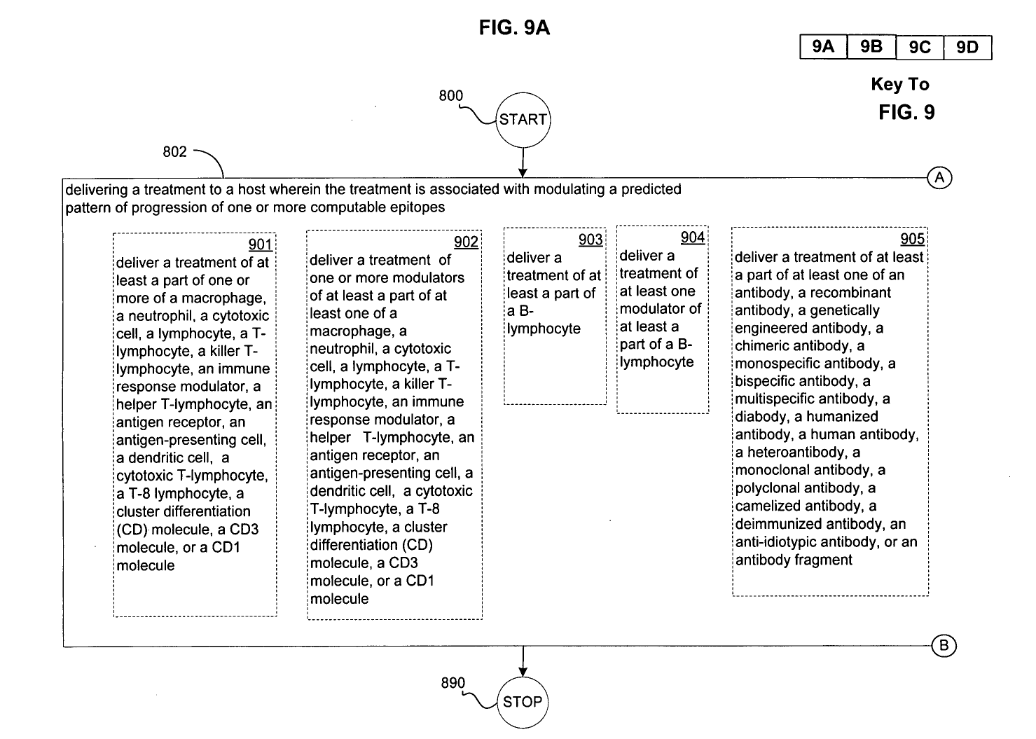

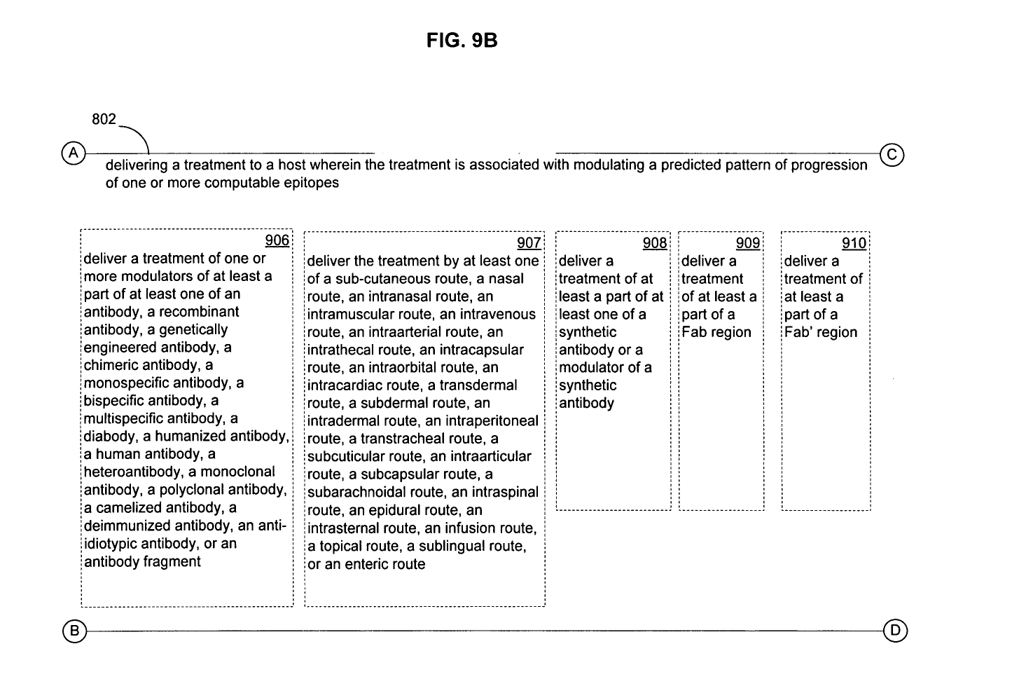

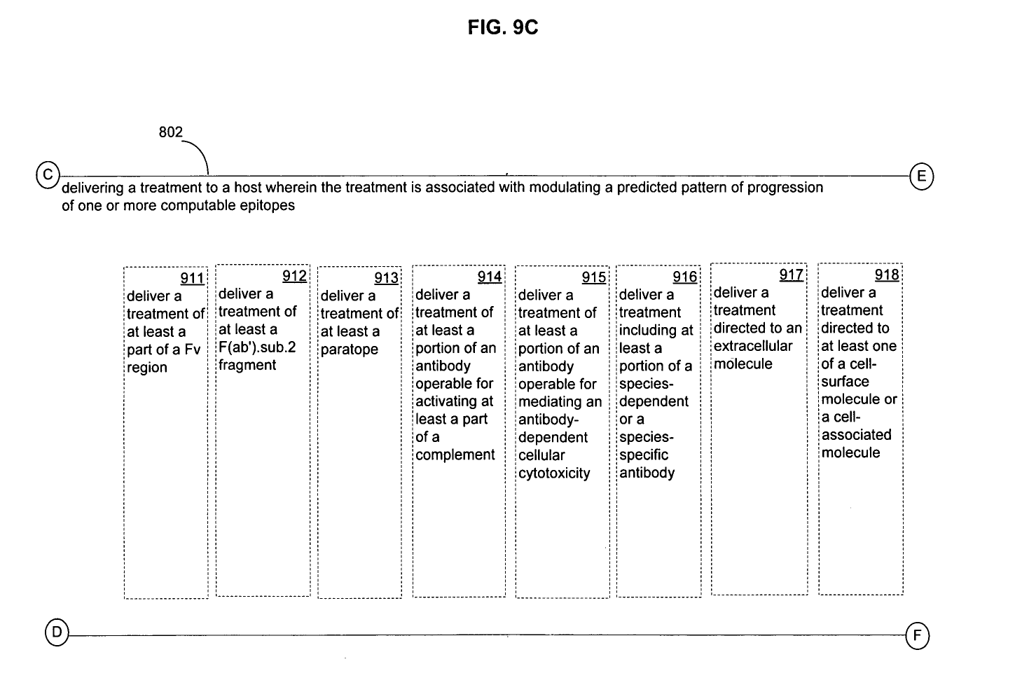

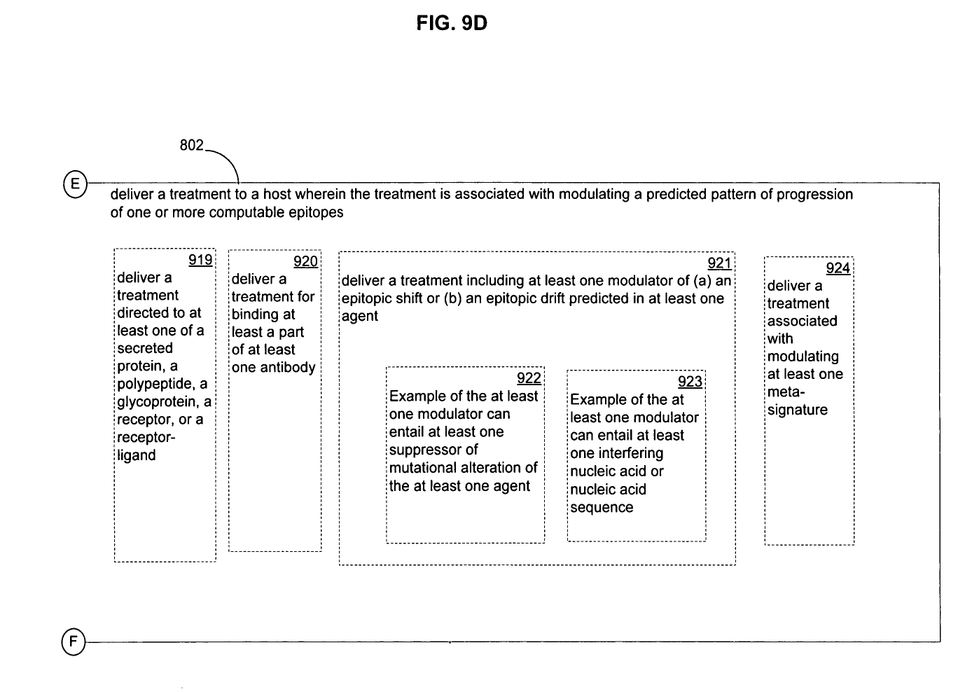

Several of the alternate process implementations are set forth herein by context. For example, as set forth herein in relation to FIG. 9D, what is described as method step 921 is illustrated as a list of exemplary qualifications of the treatment including at least one modulator of (a) an epitopic shift or (b) an epitopic drift predicted in at least one agent. Those skilled in the art will appreciate that, when what is described as example-block 922 is read in context of what is described as method step 921, it is apparent that the example set forth, in context, is actually illustrative of an alternate implementation of method step 921 of the at least one modulator of (a) an epitopic shift and/or (b) epitopic drift may include at least one suppressor of mutagenesis of the at least one agent. Likewise, as set forth herein in relation to FIG. 9D, when what is described as example-block 922 is read in the context of what are described as method step 921 and/or method step 802, it is apparent that, in context, example-block 922 is actually illustrative of an example of an alternate implementation of method step 921 and/or method step 802 of delivering a treatment including at least one modulator, such as, for example, at least one suppressor of mutagenesis of the at least one agent. Likewise again, when what is described as example-block 923 is read in the context of what are described as method step 921 and/or method step 802, it is apparent that, in context, method step 923 is actually illustrative of an alternate implementation of method step 921 and/or method step 802 of delivering a treatment including at least one modulator, such as, for example, at least one interfering nucleic acid. Contextual readings, such as those just set forth in relation to method steps 921 and/or 802, are within the ambit of one having skill in the art in light of the teaching herein, and hence are not set forth verbatim elsewhere herein for sake of clarity.

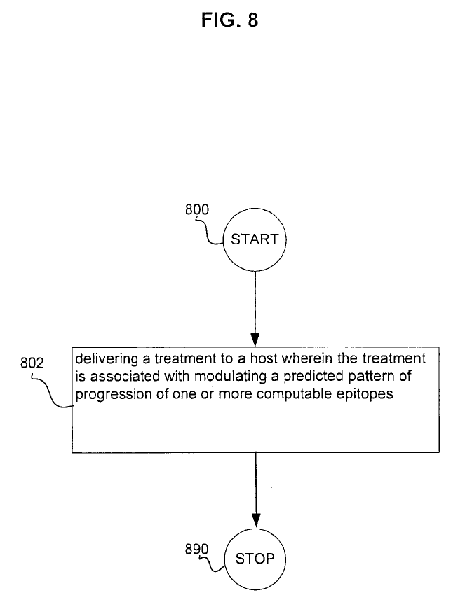

With reference now to FIG. 8, depicted is a high level logic flowchart of a process. Method step 800 shows the start of the process. Method step 802 depicts delivering a treatment to a host wherein the treatment is associated with modulating a predicted pattern of progression of one or more computable epitopes. The one or more computable epitopes may include, for example, including but not limited to, at least a portion of an agent requiring management, at least a part of at least one of an amino acid, a polypeptide, a nucleotide, a carbohydrate, a protein, a lipid, a capsid protein, a coat protein, a polysaccharide, a sugar, a lipopolysaccharide, a glycolipid, and/or a glycoprotein. It will be appreciated by those of skill in the art that the term “amino acid” may include, but is not limited to, complete and/or partial amino acids, amino acid residues, amino acid polymers and/or polypeptides, amino acid moieties, and/or components thereof. It will be appreciated by those of skill in the art that the term “nucleotide” may include, but is not limited to, complete and/or partial nucleotides, nucleotide residues, nucleotide polymers, nucleotide moieties, and/or components thereof. Method step 890 shows the end of the process. It will also be appreciated by those skilled in the art that method step 802 may include accepting input related to, for example, the agent, the one or more computable epitopes and/or other relevant criteria such as a size of the computable epitope, a type of the computable epitope, a nature of the disease, a disorder and/or a condition requiring management, and/or a sensitivity of a group requiring management.

With reference now to FIG. 9, depicted is a high-level logic flowchart depicting alternate implementations of the high-level logic flowchart of FIG. 8. Illustrated is that in various alternate implementations, method step 802 may include at least one of substeps 901, 902, 903, 904, 905, 906, 907, 908, 909, 910, 911, 912, 913, 914, 915, 916, 917, 918, 919, 990, 991, 923, and/or 924. Method step 901 depicts delivering a treatment of at least a part of one or more of a macrophage, a neutrophil, a cytotoxic cell, a lymphocyte, a T-lymphocyte, a killer T-lymphocyte, an immune response modulator, a helper T-lymphocyte, an antigen receptor, an antigen-presenting cell, a dendritic cell, a cytotoxic T-lymphocyte, a T-8 lymphocyte, a cluster differentiation (CD) molecule, a CD3 molecule, and/or a CD1 molecule. Method step 902 depicts delivering a treatment of one or more modulators of at least a part of at least one of a macrophage, a neutrophil, a cytotoxic cell, a lymphocyte, a T-lymphocyte, a killer T-lymphocyte, an immune response modulator, a helper T-lymphocyte, an antigen receptor, an antigen-presenting cell, a dendritic cell, a cytotoxic T-lymphocyte, a T-8 lymphocyte, a cluster differentiation (CD) molecule, a CD3 molecule, and/or a CD1 molecule. Method step 903 depicts delivering a treatment of at least a part of a B-lymphocyte. Method step 904 depicts delivering a treatment of at least one modulator of at least a part of a B-lymphocyte. Method step 905 depicts delivering a treatment of at least a part of at least one of an antibody, a recombinant antibody, a genetically engineered antibody, a chimeric antibody, a monospecific antibody, a bispecific antibody, a multispecific antibody, a diabody, a humanized antibody, a human antibody, a heteroantibody, a monoclonal antibody, a polyclonal antibody, a camelized antibody, a deimmunized antibody, an anti-idiotypic antibody, and/or an antibody fragment. Method step 906 depicts delivering a treatment of one or more modulators of at least a part of at least one of an antibody, a recombinant antibody, a genetically engineered antibody, a chimeric antibody, a monospecific antibody, a bispecific antibody, a multispecific antibody, a diabody, a humanized antibody, a human antibody, a heteroantibody, a monoclonal antibody, a polyclonal antibody, a camelized antibody, a deimmunized antibody, an anti-idiotypic antibody, and/or an antibody fragment. Method step 907 depicts delivering the treatment by at least one of a sub-cutaneous route, a nasal route, an intranasal route, an intramuscular route, an intravenous route, an intraarterial route, an intrathecal route, an intracapsular route, an intraorbital route, an intracardiac route, a transdermal route, a subdermal route, an intradermal route, an intraperitoneal route, a transtracheal route, a subcuticular route, an intraarticular route, a subcapsular route, a subarachnoidal route, an intraspinal route, an epidural route, an intrasternal route, an infusion route, a topical route, a sublingual route, and/or an enteric route. Method step 909 depicts delivering a treatment of at least a part of at least one of a synthetic antibody and/or a modulator of a synthetic antibody. Method step 909 depicts delivering a treatment of at least a part of a Fab region. Method step 910 depicts delivering a treatment of at least a part of a Fab′ region. Method step 911 depicts delivering a treatment of at least a part of a Fv region. Method step 912 depicts delivering a treatment of at least a F(ab′).sub.2 fragment. Method step 913 depicts delivering a treatment of at least a paratope. Method step 914 depicts delivering a treatment of at least a portion of an antibody operable for activating at least a part of a complement. Method step 915 depicts delivering a treatment of at least a portion of an antibody operable for mediating an antibody-dependent cellular cytotoxicity. Method step 916 depicts delivering a treatment including at least a portion of a species-dependent antibody and/or a species-specific antibody. Method step 917 depicts delivering a treatment directed to an extracellular molecule. Method step 918 depicts delivering a treatment directed to at least one of a cell-surface molecule and/or a cell-associated molecule. Method step 919 depicts delivering a treatment directed to at least one of a secreted protein, a polypeptide, a glycoprotein, a receptor, and/or a receptor-ligand. Method step 920 depicts delivering a treatment for binding at least a part of at least one antibody (e.g., when the immune response requiring management is an auto-immune response). Method step 921 depicts delivering a treatment including at least one modulator of (a) an epitopic shift and/or (b) an epitopic drift that is predicted in at least one agent (for example, the epitopic shift and/or drift may be computable, compositional and/or structural). The agent may include, for example, at least one of an organism, a virus, a dependent virus, an associated virus, a bacterium, a yeast, a mold, a fungus, a protoctist, an archaea, a mycoplasma, a phage, a mycobacterium, an ureaplasma, a chlamydia, a rickettsia, a nanobacterium, a prion, an agent responsible for a transmissible spongiform encephalopathy (TSE), a multicellular parasite, a protein, an infectious protein, a polypeptide, a polyribonucleotide, a polydeoxyribonucleotide, a polyglycopeptide, a nucleic acid, an infectious nucleic acid, a polymeric nucleic acid, a metabolic byproduct, a cellular byproduct, and/or a toxin. Method step 924 depicts delivering a treatment associated with modulating at least one meta-signature (e.g., a consensus sequence).

With reference now to FIG. 9D, depicted is a high-level logic flowchart depicting alternate implementations of the high-level logic flowchart of FIG. 8. Shown is various alternate implementation, method step 921 may include at least one of example-blocks 922 and/or 923 which further sets forth the at least one modulator. Example-block 922 depicts that examples of delivering a treatment may include at least one suppressor of mutational alteration of the at least one agent (e.g., an escape mutation for down-regulating and/or up-regulating a gene and/or a related gene activity). Example block 923 depicts that examples of delivering a treatment may include at least one interfering nucleic acid and/or nucleic acid sequence (e.g., one or more of a deoxynucleotide, a chemically synthesized nucleotide, a nucleotide analog, a nucleotide not naturally occurring, or a nucleotide not found in natural RNA or DNA of an untreated agent and/or a (e.g., polymerized) set of such nucleic acids).

C. Variation(s), and/or Implementation(s)

Those having skill in the art will recognize that the present application teaches modifications of the devices, structures, and/or processes within the spirit of the teaching herein. For example, in one aspect, the immune response components may be formulated to cross the blood-brain barrier which is known to exclude mostly hydrophilic compounds, as well as to discriminate against transport of high molecular weight ones. For example, an antibody fragment may be encased in a lipid vesicle. In another example, the antibody or a portion of the antibody may be tagged onto a carrier protein or molecule. In another example, an antibody or other immune response component may be split into one or more complementary fragments, each fragment encased by a lipid vesicle, and each fragment functional only on binding its complementary fragment. Once the blood-brain barrier has been traversed, the lipid vesicle may be dissolved to release the antibody fragments which reunite with their complementary counterparts and may form a fully functional antibody or other immune response component. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, the immune response components may be made in large format. The method lends itself to both small format or personalized care applications and large-scale applications or large format applications. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, the method may be used to designate immune response components for any disease or disorder. The application of this method is not limited to diseases where antigenic shift or drift keeps the immune system “guessing” causing it to be effectively slow-to-respond or to be incapable of effective and/or definitive (e.g., infection-clearing) response. Although, influenza-A or HIV-1 are likely viral-disease-agent candidates for application of this method, treatment of other diseases, disorders and/or conditions will likely benefit from this methodology. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, real-time evaluation may be provided of the antigenic changes by including a portable PCR-enabled machine which samples the environment for (sub-)strains of pathogens locally present. The information may be sent remotely to another location or to a portable material-administering device, for example, a drip-patch device with a remote sensor, utilized by a potentially-affected host, resulting in the activation of the necessary immune response components and thereby providing adequate immuno-protection to the potential host. As the evaluation possibly changes in time, the portable material-administering material-device may be controlled to change the dosage or type of immune response component delivered. Such a portable material-administering device operably coupled to a portable PCR-enabled machine or a fully functional system has a wide variety of applications, for example, including, but not limited to, when medical personnel visit an area in which a disease is endemic, and/or when military or security personnel enter territory in which unknown pathogens may be present. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, a potential host may use a material-administering device including the immune response components preprogrammed to provide the potential host with the necessary immune response-mediated protection over an interval of time, and/or to anticipate pattern changes in the epitopes of the agent 100. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, RNA blockers, and/or RNAi technology may be used to down-regulate genes or interfere productively with their expression, or to otherwise usefully modulate components of the immune system in conjunction with the method. Other modifications of the subject matter herein will be appreciated by one of skill in the art in light of the teachings herein.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency vs. operational convenience tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently and universally superior to any other, in that any vehicle to be utilized is a choice dependent upon the context in which the vehicle will be deployed and the specific concerns (e.g., speed, flexibility, or predictability) of the implementer, any of which may vary substantially.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), other integrated formats, or other extensively-integrated formats. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter subject matter described herein applies equally regardless of the particular type of signal-bearing media used to actually carry out the distribution. Examples of a signal-bearing media include, but are not limited to, the following: recordable type media such as floppy disks, hard disk drives, DVD/CD-ROMs, digital tape, computer memory devices of various types; and data transmission type-media such as digital and analog communication links using TDM or IP-based communication links (e.g., packetized data links).

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application-specific integrated circuit, electrical circuitry forming a general-purpose computing device configured by a computer program (e.g., a general-purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment).

Those skilled in the art will recognize that it is common within the art to describe devices and/or processes in the fashion set forth herein, and thereafter use standard engineering practices to integrate such described devices and/or processes into data-processing systems. That is, at least a portion of the devices and/or processes described herein can be integrated into a data-processing system via a reasonable amount of experimentation. Those having skill in the art will recognize that a typical data processing system generally includes one or more of a system unit housing, a display device, a video display device, a memory such as volatile and/or non-volatile memory, processors such as microprocessors and digital signal processors, computational entities such as operating systems, drivers, user interfaces (e.g. graphical), and applications programs, one or more interaction devices, such as a touch-pad or screen, and/or control systems including feedback loops and control motors (e.g., feedback for sensing position and/or velocity; control motors for moving and/or adjusting components such as valves and/or quantities). A typical data processing system may be implemented utilizing any suitable commercially available components, such as those typically found in digital data computing/communication and/or network computing/communication systems.

All of the referenced U.S. patents, U.S. patent application publications, U.S. patent applications, foreign patents, foreign patent applications, and/or non-patent publications referred to in this specification and/or listed in any Application Data Sheet, are incorporated herein by reference, in their entireties.

The herein described aspects depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components.

While particular aspects of the present subject matter described herein have been shown and described, it will be apparent to those skilled in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.).

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. A system comprising:
circuitry for suggesting delivery of a treatment to a host wherein the treatment is associated with modulating a predicted pattern of progression of one or more computable epitopes.

27. The system of claim 26, wherein the circuitry for suggesting delivery of a treatment further comprises:
circuitry for suggesting delivery of a treatment of at least a part of one or more of a macrophage, a neutrophil, a cytotoxic cell, a lymphocyte, a T-lymphocyte, a killer T-lymphocyte, an immune response modulator, a helper T-lymphocyte, an antigen receptor, an antigen-presenting cell, a dendritic cell, a cytotoxic T-lymphocyte, a T-8 lymphocyte, a cluster differentiation (CD) molecule, a CD3 molecule, or a CD1 molecule.

28. The system of claim 26, wherein the circuitry for suggesting delivery of a treatment further comprises:
circuitry for suggesting delivery of a treatment of one or more modulators of at least a part of at least one of a macrophage, a neutrophil, a cytotoxic cell, a lymphocyte, a T-lymphocyte, a killer T-lymphocyte, an immune response modulator, a helper T-lymphocyte, an antigen receptor, an antigen-presenting cell, a dendritic cell, a cytotoxic T-lymphocyte, a T-8 lymphocyte, a cluster differentiation (CD) molecule, a CD3 molecule, or a CD1 molecule.

29. The system of claim 26, wherein the circuitry for suggesting delivery of a treatment further comprises:
circuitry for suggesting delivery of a treatment of at least a part of a B-lymphocyte.

30. The system of claim 26, wherein the circuitry for suggesting delivery of a treatment further comprises:
circuitry for suggesting delivery of a treatment of at least one modulator of at least a part of a B-lymphocyte.

31. The system of claim 26, wherein the circuitry for suggesting delivery of a treatment further comprises:
circuitry for suggesting delivery of a treatment of at least a part of at least one of an antibody, a recombinant antibody, a genetically engineered antibody, a chimeric antibody, a monospecific antibody, a bispecific antibody, a multispecific antibody, a diabody, a chimeric antibody, a humanized antibody, a human antibody, a heteroantibody, a monoclonal antibody, a polyclonal antibody, a camelized antibody, a deimmunized antibody, an anti-idiotypic antibody, or an antibody fragment.

32. The system of claim 26, wherein the circuitry for suggesting delivery of a treatment further comprises:
circuitry for suggesting delivery of a treatment of one or more modulators of at least a part of at least one of an antibody, a recombinant antibody, a genetically engineered antibody, a chimeric antibody, a monospecific antibody, a bispecific antibody, a multispecific antibody, a diabody, a chimeric antibody, a humanized antibody, a human antibody, a heteroantibody, a monoclonal antibody, a polyclonal antibody, a camelized antibody, a deimmunized antibody, an anti-idiotypic antibody, or an antibody fragment.

33. The system of claim 26, wherein the circuitry for suggesting delivery of a treatment further comprises:
circuitry for suggesting delivery of a treatment by at least one of a sub-cutaneous route, a nasal route, an intranasal route, an intramuscular route, an intravenous route, an intraarterial route, an intrathecal route, an intracapsular route, an intraorbital route, an intracardiac route, a transdermal route, a subdermal route, an intradermal route, an intraperitoneal route, a transtracheal route, a subcuticular route, an intraarticular route, a subcapsular route, a subarachnoidal route, an intraspinal route, an epidural route, an intrasternal route, an infusion route, a topical route, a sublingual route, or an enteric route.

34. The system of claim 26, wherein the circuitry for suggesting delivery of a treatment further comprises:
circuitry for suggesting delivery of a treatment of at least a part of at least one of a synthetic antibody or a modulator of a synthetic antibody.

35. The system of claim 26, wherein the circuitry for suggesting delivery of a treatment further comprises:
circuitry for suggesting delivery of a treatment of at least a part of a Fab region.

36. The system of claim 26, wherein the circuitry for suggesting delivery of a treatment further comprises:
circuitry for suggesting delivery of a treatment of at least a part of a Fab′ region.

37. The system of claim 26, wherein the circuitry for suggesting delivery of a treatment further comprises:
circuitry for suggesting delivery of a treatment of at least a part of a Fv region.

38. The system of claim 26, wherein the circuitry for suggesting delivery of a treatment further comprises:
circuitry for suggesting delivery of a treatment of at least a F(ab′).sub.2 fragment.

39. The system of claim 26, wherein the circuitry for suggesting delivery of a treatment further comprises:
circuitry for suggesting delivery of a treatment of at least a paratope.

40. The system of claim 26, wherein the circuitry for suggesting delivery of a treatment further comprises:
circuitry for suggesting delivery of a treatment of at least a portion of an antibody operable for activating at least a part of a complement.

41. The system of claim 26, wherein the circuitry for suggesting delivery of a treatment further comprises:
circuitry for suggesting delivery of a treatment of at least a portion of an antibody operable for mediating an antibody-dependent cellular cytotoxicity.

42. The system of claim 26, wherein the circuitry for suggesting delivery of a treatment further comprises:
circuitry for suggesting delivery of a treatment including at least a portion of a species-dependent antibody or a species-specific antibody.

43. The system of claim 26, wherein the circuitry for suggesting delivery of a treatment further comprises:
circuitry for suggesting delivery of a treatment directed to an extracellular molecule.

44. The system of claim 26, wherein the circuitry for suggesting delivery of a treatment further comprises:
circuitry for suggesting delivery of a treatment directed to at least one of a cell-surface molecule or a cell-associated molecule.

45. The system of claim 26, wherein the circuitry for suggesting delivery of a treatment further comprises:
circuitry for suggesting delivery of a treatment directed to at least one of a secreted protein, a polypeptide, a glycoprotein, a receptor, or a receptor-ligand.

46. The system of claim 26, wherein the circuitry for suggesting delivery of a treatment further comprises:
circuitry for suggesting delivery of a treatment for binding at least a part of at least one antibody.

47. The system of claim 26, wherein the circuitry for suggesting delivery of a treatment further comprises:
circuitry for suggesting delivery of a treatment including at least one modulator of (a) an epitopic shift or (b) an epitopic drift predicted in at least one agent.

48. The system of claim 47, wherein the circuitry for suggesting delivery of a treatment including at least one modulator of (a) an epitopic shift or (b) an epitopic drift predicted in at least one agent further comprises:
at least one suppressor of mutational alteration of the at least one agent.

49. The system of claim 47, wherein the circuitry for suggesting delivery of a treatment including at least one modulator of (a) an epitopic shift or (b) an epitopic drift predicted in at least one agent further comprises:
at least one interfering nucleic acid or nucleic acid sequence.

50. The system of claim 26, wherein the circuitry for suggesting delivery of a treatment further comprises:
circuitry for delivering a treatment associated with modulating at least one meta-signature.

51. (canceled)

52. (canceled)

53. (canceled)

54. (canceled)

55. (canceled)

56. (canceled)

57. (canceled)

58. A system, comprising:
means for suggesting delivery of a treatment to a host wherein the treatment is associated with modulating a predicted pattern of progression of one or more computable epitopes.

59. A method, comprising:
automatically suggesting delivery of a treatment to a host wherein the treatment is associated with modulating a predicted pattern of progression of one or more computable epitopes.
